SEQUENCE LISTING (12 sequences)

SEQ ID NO: 1 - protein, 7 aa, synthetic construct
  VARIANT 1: note = X = A, D, E, F, G, H, I, K, L, M, N, P, Q, R, S, T, V, W, or Y
  VARIANT 5: note = X = A, D, E, F, G, H, I, K, L, M, N, P, Q, R, S, T, V, W, or Y
XDGRXGF

SEQ ID NO: 2 - protein, 7 aa, synthetic construct
QDGRMGF

SEQ ID NO: 3 - protein, 7 aa, synthetic construct
QDGRKGF

SEQ ID NO: 4 - protein, 8 aa, synthetic construct
QDGRKGGF

SEQ ID NO: 5 - protein, 9 aa, synthetic construct
  VARIANT 2: note = X = A, D, E, F, G, H, I, K, L, M, N, P, Q, R, S, T, V, W, or Y
  VARIANT 6: note = X = A, D, E, F, G, H, I, K, L, M, N, P, Q, R, S, T, V, W, or Y
CXDGRXGFC

SEQ ID NO: 6 - protein, 9 aa, synthetic construct
CQDGRMGFC

SEQ ID NO: 7 - protein, 9 aa, synthetic construct
QHWSYGLRP

SEQ ID NO: 8 - protein, 381 aa, synthetic construct
MKDLTVLSRWRYSWRILDDNEAQKMKKTAIAIAVALAGFATVAQAAPKDNTWYAGAKLGWSQYHDTGFINNDGPTHENQLGAGAFGGYQVNPYVGFEMGYDWLGRMPYKGDNINGAYKAQGVQLTAKLGYPITDDLDVYTRLGGMVWRADTKSNVPGQDGRMGFGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 9 - protein, 381 aa, synthetic construct
MKDLTVLSRWRYSWRILDDNEAQKMKKTAIAIAVALAGFATVAQAAPKDNTWYAGAKLGWSQYHDTGFINNDGPTHENQLGAGAFGGYQVNPYVGFEMGYDWLGRMPYKGDNINGAYKAQGVQLTAKLGYPITDDLDVYTRLGGMVWRADTKSNVPGQDGRKGFGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 10 - protein, 382 aa, synthetic construct
MKDLTVLSRWRYSWRILDDNEAQKMKKTAIAIAVALAGFATVAQAAPKDNTWYAGAKLGWSQYHDTGFINNDGPTHENQLGAGAFGGYQVNPYVGFEMGYDWLGRMPYKGDNINGAYKAQGVQLTAKLGYPITDDLDVYTRLGGMVWRADTKSNVPGQDGRKGGFGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 11 - protein, 383 aa, synthetic construct
MKDLTVLSRWRYSWRILDDNEAQKMKKTAIAIAVALAGFATVAQAAPKDNTWYAGAKLGWSQYHDTGFINNDGPTHENQLGAGAFGGYQVNPYVGFEMGYDWLGRMPYKGDNINGAYKAQGVQLTAKLGYPITDDLDVYTRLGGMVWRADTKSNVPGCQDGRMGFCGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 12 - protein, 383 aa, synthetic construct
MKDLTVLSRWRYSWRILDDNEAQKMKKTAIAIAVALAGFATVAQAAPKDNTWYAGAKLGWSQYHDTGFINNDGPTHENQLGAGAFGGYQVNPYVGFEMGYDWLGRMPYKGDNINGAYKAQGVQLTAKLGYPITDDLDVYTRLGGMVWRADTKSNVPGQHWSYGLRPGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

(19) United States
(12) Patent Application Publication
(10) Pub. No.: US 2026/0364123 A1
(43) Pub. Date: Nov. 19, 2026

(54) CANCER-SPECIFIC DELIVERY OF BACTERIUM

(71) Applicant: Chong-xian PAN, Newton, MA (US)

(72) Inventor: Chong-xian PAN, Newton, MA (US)

(21) Appl. No.: 19/118,562

(22) PCT Filed: Oct. 5, 2023
(86) PCT No.: PCT/US2023/076104

§ 371 Date: Apr. 4, 2025

Related U.S. Application Data
(60) Provisional application No. 63/378,660, filed on Oct. 6, 2022.

(51) Int. Cl. A61K 35/74; A61K 9/00; A61K 35/02; A61K 38/19; A61K 38/46; A61K 39/395; A61K 47/64; A61K 47/69; A61P 35/00

(52) U.S. Cl. CPC A61K 35/74; A61K 9/0053; A61K 38/195; A61K 38/465; A61K 39/3955; A61K 47/64; A61K 47/6901; A61P 35/00; A61K 35/02

(57) ABSTRACT

The present invention provides a method of delivering a tumor-targeting bacterium to a tumor site in an individual having cancer, comprising orally administering to the individual an effective amount of the tumor-targeting bacterium or bacteria. The tumor-targeting bacterium comprises on its surface a tumor-targeting agent so that upon administration the tumor-targeting bacterium is specifically targeted to the tumor site. The tumor-targeting bacterium can be used for treating cancer, or for the purpose of delivering therapeutic or diagnostic agents to a tumor site.

### CROSS REFERENCE TO RELATED APPLICATIONS

This patent application claims priority benefit of U.S. Provisional Application No. 63/378,660 filed on Oct. 6, 2022, the content of which is incorporated herein by reference in its entirety.

### REFERENCE TO AN ELECTRONIC SEQUENCE LISTING

The content of the electronic sequence listing (267292000140seqlist.xml; Size: 14,974 bytes; and Date of Creation: Sep. 29, 2023) is herein incorporated by reference in its entirety.

### FIELD OF THE INVENTION

The present application relates to compositions and methods for delivery of tumor-targeting bacteria to cancer cells.

### BACKGROUND

Approximately 100 trillion bacteria normally exist in the digestive system. Intestinal mucosa constitutes physical, biochemical and immune barriers that prevent bacteria inside the digestive system from entering into the body and going to other organs. Hence, bacteria in the gut usually do not enter into the blood circulation and cause disease. One study showed that oral bacteria could reach metastatic cancer liver, but only approximately 10⁶ colony-forming unit (CFU or live bacteria) per gram of tissue, or one bacterium per several hundreds of cancer cells (Danino et al. Sci Transl Med. 2015; 7: 289ra284).

The disclosures of all publications, patents, patent applications and published patent applications referred to herein are hereby incorporated herein by reference in their entirety.

### SUMMARY

In some aspects, provided herein is a method of delivering a tumor-targeting bacterium to a tumor site in an individual having cancer, comprising orally administering to the individual an effective amount of the tumor-targeting bacterium, wherein the tumor-targeting bacterium comprises on its surface a tumor-targeting agent, wherein upon administration the tumor-targeting bacterium is specifically targeted to the tumor site.

In some aspects, provided herein is a method of delivering a therapeutic or diagnostic agent to a tumor site in an individual having cancer, comprising orally administering to the individual an effective amount of the tumor-targeting bacterium, wherein the tumor-targeting bacterium comprises on its surface a tumor-targeting agent, wherein the tumor-targeting bacterium further comprises the therapeutic or diagnostic agent or a nucleic acid encoding the therapeutic or diagnostic agent, wherein upon administration the tumor-targeting bacterium is specifically targeted to the tumor site, thereby delivering the therapeutic or diagnostic agent to the tumor site.

In some of any of the provided embodiments, the tumor-targeting agent is a tumor-targeting peptide or tumor-targeting protein. In some of any of the provided embodiments, the tumor-targeting bacterium is genetically engineered to express the tumor-targeting peptide or tumor-targeting protein on its surface. In some of any of the provided embodiments, the tumor-targeting agent is attached to the surface of the tumor-targeting bacterium. In some of any of the provided embodiments, the tumor-targeting peptide is no longer than 25 amino acids in length.

In some of any of the provided embodiments, the tumor-targeting peptide is selected from the group consisting of a PLZ peptide and a luteinizing hormone releasing hormone (LHRH) peptide.

In some of any of the provided embodiments, the tumor-targeting peptide comprises the amino acid sequence X₁DGRX₅GF (SEQ ID NO:1), wherein X₁ is selected from Gln and Gly and X₅ is any amino acid other than cysteine.

In some of any of the provided embodiments, the tumor-targeting peptide comprises the amino acid sequence CX₁DGRX₅GFC (SEQ ID NO:5), wherein X₁ is selected from Gln and Gly and X₅ is any amino acid other than cysteine.

In some of any of the provided embodiments, the tumor-targeting peptide is embedded in a bacterial protein presented at the outer wall of the tumor-targeting bacterium.

In some of any of the provided embodiments, the tumor-targeting bacterium is Salmonella or Listeria.

In some of any of the provided embodiments, the therapeutic or diagnostic agent is selected from the group consisting of: a protein (such as chemokine e.g., CXCL11), an antibody, an mRNA, an siRNA, an antisense RNA, and a CRISPR/gRNA system. the therapeutic agent is a toxin or a photosensitizer.

In some of any of the provided embodiments, the therapeutic agent is an immune check point inhibitor, optionally selected from the group consisting of PD1-Fc, PD-L1-Fc, anti-PD1 antibody, and an anti-PD-L1 antibody.

In some of any of the provided embodiments, the tumor-targeting bacterium is administered at about 1×10⁷ to 5×10¹² Colony forming units (CFU) per dose.

In some of any of the provided embodiments, the tumor-targeting bacterium is administered one to four times daily. In some of any of the provided embodiments, the tumor-targeting bacterium is administered for at least a year.

In some of any of the provided embodiments, the individual has breast cancer, prostate cancer, or bladder cancer.

In some of any of the provided embodiments, the individual has a healthy gut microbiome.

In some of any of the provided embodiments, the individual is a human individual.

In some aspects, provided herein is an oral formulation of tumor-targeting bacterium comprising on its surface a tumor-targeting agent and a pharmaceutically acceptable excipient. In some of any of the provided embodiments, the tumor-targeting bacterium further comprises a therapeutic or diagnostic agent or a nucleic acid encoding a therapeutic or diagnostic agent. In some of any of the provided embodiments, the oral formulation is in the form of capsule or liquid formulation.

In some of any of the provided embodiments of the oral formulation, the tumor-targeting agent is a tumor-targeting peptide or tumor-targeting protein. In some of any of the provided embodiments, the tumor-targeting bacterium is genetically engineered to express the tumor-targeting peptide or tumor-targeting protein on its surface. In some of any of the provided embodiments, the tumor-targeting agent is attached to the surface of the tumor-targeting bacterium. In some of any of the provided embodiments, the tumor-targeting peptide is no longer than 25 amino acids in length.

In some of any of the provided embodiments of the oral formulation, the tumor-targeting peptide is selected from the group consisting of a PLZ peptide and a luteinizing hormone releasing hormone (LHRH) peptide.

In some of any of the provided embodiments of the oral formulation, the tumor-targeting peptide comprises the amino acid sequence X₁DGRX₅GF (SEQ ID NO:1), wherein X₁ is selected from Gln and Gly and X₅ is any amino acid other than cysteine.

In some of any of the provided embodiments of the oral formulation, the tumor-targeting peptide comprises the amino acid sequence CX₁DGRX₅GFC (SEQ ID NO:5), wherein X₁ is selected from Gln and Gly and X₅ is any amino acid other than cysteine.

In some of any of the provided embodiments of the oral formulation, the tumor-targeting peptide is embedded in a bacterial protein presented at the outer wall of the tumor-targeting bacterium.

In some of any of the provided embodiments of the oral formulation, the tumor-targeting bacterium is Salmonella or Listeria.

In some of any of the provided embodiments of the oral formulation, the therapeutic or diagnostic agent is selected from the group consisting of: a protein (such as chemokine e.g., CXCL11), an antibody, an mRNA, an siRNA, an antisense RNA, and a CRISPR/gRNA system, the therapeutic agent is a toxin or a photosensitizer.

In some of any of the provided embodiments of the oral formulation, the therapeutic agent is an immune check point inhibitor, optionally selected from the group consisting of PD1-Fc, PD-L1-Fc, anti-PD1 antibody, and an anti-PD-L1 antibody.

In some of any of the provided embodiments, the oral formulation comprises one or more other bacteria. In some of any of the provided embodiments, the one or more other bacteria comprises tumor-targeting bacteria, the one or more bacteria comprises bacteria which are not tumor-targeting bacteria.

### BRIEF DESCRIPTION OF THE DRAWINGS

FIG. 1A shows bacterial numbers in Colony Forming Unites (CFU)/g tissue at 24, 48, 72 and 96 hours after administration of 5×10⁸ CFU PLZ4-Salmonella-haPD1-EGFP in samples from the indicated tissues. Tissues were weighed, homogenized and cultured for bacterial numbers. FIG. 1B shows actual colony numbers per gram of tissue at 24, 48, 72 and 96 hours after administration. The colonies of bacteria at tumor were 6,208, 6,123, 920 and 80 times higher than that in the liver at 24, 48, 72 and 96 hours after administration, respectively. The Salmonella used in this study had self-limited lifespan, so colonies at 96 hours were only 0.4% of the colonies at 72 hours.

FIG. 2 shows cancer-specific delivery of PLZ4-Salmonella-KillerRed. A SV40T/Ras double transgenic mouse and a control mouse were fed with PLZ4-Salmonella-expressing KillerRed at 5×10⁸ CFU per mouse at 72 hours before mice was euthanized. Bladder and vital organs were harvested, homogenized and cultured for PLZ4-Salmonella.

FIG. 2A (left plot) shows a bar graph showing PLZ4-Salmonella-KillerRed colony numbers in CFU/g tissue in bladder cancer and vital organs of the SV40T/Ras double transgenic mouse. FIG. 2B (right plot) shows a bar graph showing PLZ4-Salmonella-KillerRed colony numbers in CFU/g tissue in the bladder and vital organs of control mice. FIG. 2C shows actual numbers of PLZ4-Salmonella-KillerRed in CFU/g tissue in SV40T/Ras and control mice.

FIGS. 3A and 3B shows targeting of Salmonella lacking the targeting peptide PLZ4. FIG. 3A shows colony numbers from the indicated organs of mice orally administered 5×10⁸ CFU Salmonella. FIG. 3B shows colony numbers from the indicated organs of mice intravenously administered 10⁸ CFU Salmonella.

FIG. 4 shows targeting of tumors based on KillerRed expression at tumor sites. mice were fed with 5×10⁸ CFU of PLZ4-Salmonella that express KillerRed, a red fluorescent protein. Tumors and organs were harvested at 24, 48, 72 and 96 hours after oral administration. Tumors were cut in half in order to show the exterior and interior of each tumor, as indicated above the image.

FIG. 5 shows expression of KillerRed from PLZ4-Salmonella in orthotopic bladder cancer tumors. Organs were harvested at 72 hours after oral administration of PLZ4-Salmonella-KillerRed.

FIG. 6 shows expression of haPD1-EGFP from PLZ4-Salmonella-haPD1-EGFP in C57BL/6 mice fed with 5×10⁸ CFU of PLZ4-Salmonella expressing haPD1-EGFP. Subcutaneous tumors were harvested at 24, 48, 72 and 96 hours before euthanasia.

FIG. 7 shows reactive oxygen species (ROS) production from PLZ4-Salmonella-KellerRed cells following photodynamic therapy (PDT). C57BL/6 mice carrying UPPL syngeneic bladder cancers were fed with PLZ4-Salmonella-KellerRed at 5×10⁸ CFU per mouse. After 72 hours, laser (wavelength: 561 nm; 100 mW for 20 minutes) was applied to the tumor for PDT. After 24 hours, tumors from both mice were harvested, fresh frozen, cut at 30 μm thick, and stained with dihydroethydine (DHE) at 10 μmol/L dissolved in DMSO, and incubated in light-protected humidified chamber at 37 °C for 15 minutes. Green fluorescence indicates ROS production.

FIGS. 8A and 8B shows T cell infiltration into tumors following PDT in mice treated with PLZ4-Salmonella-CXCL11 and PLZ4-Salmonella-KillerRed. FIG. 8A shows flow cytometry analysis of cells in tumors. The left panel is a control panel, the middle panel is tumor sample from a mouse treated with PLZ4-Salmonella-CXCL11, and the right panel is a tumor panel from a mouse treated with PLZ4-Salmonella-KillerRed plus photodynamic therapy. FIG. 8B shows CD8 T cells in tumors. In the control tumors, only 0.8% of all cells were CD8 T cells. In the PLZ4-Salmonella-CXCL11 group, 9.5% of all cells were CD8 T cells. In the PLZ4-Salmonella-KillerRed plus photodynamic therapy group, 22.5% cells were T cells.

FIG. 9 shows anti-tumor activity of PLZ4-Salmonella-haPD1-IgG2a. C57BL/6 mice carrying syngeneic BBN963 tumors were randomized to receive different treatment as indicated in the figure. Mice received 5×10⁸ CFU via oral gavage.

FIG. 10A shows cancer-specific homing of PLZ4-Salmonella expressing KillerRed after prior immunization. C57BL/6 mice were immunized with Salmonella on Day 1, 4, 22, 24, 36 and 38 before inoculation of bladder cancer cells BBN cells into the flank. When tumor sizes reached around 1.5 cm³, mice were fed 5×10⁸ CFU PLZ4-Salmonella-KillerRed, and sacrificed 72 hours later. FIG. 10A shows light and fluorescent imaging showing KillerRed expression. FIG. 10B shows Salmonella CFU per gram of tissue in the different organs and tumors.

FIG. 11 shows cancer-specific targeting of LHRH-Salmonella in mice fed 0.36×10⁸ CFU of LHRH-Salmonella. Tumors and tissues were collected 72 hours after feeding.

FIG. 12A shows the anti-cancer effects of PLZ4-Salmonella-haPD1-IgG2a. C57BL/6 mice carrying syngeneic BBN963 tumors were randomized to receive one of the following treatments: PBS; control Salmonella 10⁹ CFU oral gavage once weekly; PLZ4-Salmonella-haPD1-IgG2a 10⁹ CFU oral gavage once weekly; or anti-PD1 antibody at 200 μg intravenous once weekly. The tumor ratio was measured over time. FIG. 12B shows the p values for data points corresponding to PLZ4-Salmonella-haPD1 vs PBS and Anti-PD1 antibody vs PBS on Days 4, 7, 10, and 14.

FIG. 13A shows a graph quantifying Listeria colonies from tumors and various organs of mice fed with 10⁸ CFU of control Listeria. FIG. 13B shows a graph quantifying Listeria colonies from tumors and various organs of mice fed with 10⁸ CFU of PLZ4-Listeria. FIG. 13C shows a table listing the number of Listeria colonies from tumors and various organs of mice fed with 10⁸ CFU of control Listeria. FIG. 13D shows a table listing the number of Listeria colonies from tumors various organs of mice fed with 10⁸ CFU of PLZ4-Listeria. Mice were euthanized and organs were harvested around 72 hours after feeding. Three mice were used for each condition and tumor or organ.

### DETAILED DESCRIPTION

Provided herein are methods of delivering (e.g., by orally administering) a tumor-targeting bacterium to a tumor site in an individual having cancer and tumor-targeting bacterium useful for such purposes. The tumor-targeting bacterium comprises on its surface a tumor-targeting agent which allows the tumor-targeting bacterium to be specifically targeted to a tumor site upon administration (e.g., oral administration).

The present application is based on the surprising finding that a tumor-targeting agent (e.g., a tumor-targeting peptide or protein) present on the surface of a bacteria (e.g., Salmonella or Listeria) can specifically guide the delivery of the bacteria to the tumor site after oral administration. Such targeting can be achieved even when the subject was previously immunized with the bacteria. The tumor-targeting bacteria can bring along therapeutic agents (or diagnostic agents) associated with them (e.g., therapeutic proteins expressed by the bacteria), allowing high concentration of the therapeutic agent (or diagnostic agents) at the tumor site to achieve therapeutic results.

The tumor-targeting bacteria and corresponding delivery methods provided herein open a new and exciting opportunity of delivering to a tumor site therapeutic and/or diagnostic agents, including protein/peptide-based drugs and gene therapy agents, though oral administration. Because many biologic drugs (such as proteins and peptides) can be destroyed at a gastrointestinal tract, they are usually administered to patients through parenteral infusion or injection. The tumor-targeting bacteria described herein make it possible to administer therapeutic and/or diagnostic agents, including biologic drugs, via oral administration. Unlike many biologic drugs (such as protein and peptide drugs) which are unstable and require specific conditions for storage and transportation, the tumor-targeting bacteria described herein are much more stable and can be stored and/or transported at 4 °C or room temperature. In addition, culturing and amplification of bacteria are cheaper and more straightforward, and would avoid the long and expensive process of biologic production and purification process during manufacturing. Moreover, because of high tumor site specificity of the tumor-targeting bacteria, the therapeutic and/or diagnostic agents can be specifically delivered to the target tumor site without causing undesirable, non-specific toxicity.

Additionally, the tumor-targeting bacteria described herein themselves can stimulate immune response, and can either be effective in treating cancer on their own, or be useful for potentiating the anticancer effects of other therapeutic agents (such as immunotherapy).

Thus, the present application in one aspect provides a method of delivering a tumor-targeting bacterium to a tumor site in an individual having cancer, comprising orally administering to the individual an effective amount of the tumor-targeting bacterium or bacteria, wherein the tumor-targeting bacterium comprises on its surface a tumor-targeting agent, wherein upon administration the tumor-targeting bacterium is specifically targeted to the tumor site.

In another aspect, there is provided a method of delivering a therapeutic or diagnostic agent to a tumor site in an individual having cancer, comprising orally administering to the individual an effective amount of the tumor-targeting bacterium, wherein the tumor-targeting bacterium comprises on its surface a tumor-targeting agent, wherein the tumor-targeting bacterium further comprises the therapeutic or diagnostic agent, or a nucleic acid encoding the therapeutic or diagnostic agent, wherein upon administration the tumor-targeting bacterium is specifically targeted to the tumor site, thereby delivering the therapeutic or diagnostic agent to the tumor site.

In another aspect, there are provided methods of treating cancer by delivering the tumor-targeting bacteria as described above.

In another aspect, there are provided oral formulations of a tumor-targeting bacterium comprising on its surface a tumor-targeting agent and a pharmaceutically acceptable excipient.

### DEFINITIONS

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The terms “subject,” “individual,” and “patient” are used interchangeably herein to refer to a mammal, including, but not limited to, human, bovine, horse, feline, canine, rodent, or primate. In some embodiments, the individual is a human.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof.

Reference to “about” a value or parameter herein includes (and describes) variations that are directed to that value or parameter per se. For example, description referring to “about X” includes description of “X”.

The term “about X-Y” used herein has the same meaning as “about X to about Y.”

As used herein and in the appended claims, the singular forms “a,” “or,” and “the” include plural referents unless the context clearly dictates otherwise.

### METHODS OF THE PRESENT INVENTION

The present application in one aspect provides a method of delivering a tumor-targeting bacterium (e.g., Salmonella, such as Salmonella typhimurium, or Listeria, such as Listeria monocytogenes) to a tumor site in an individual having cancer, comprising orally administering to the individual an effective amount of the tumor-targeting bacterium, wherein the tumor-targeting bacterium comprises on its surface a tumor-targeting agent (such as tumor-targeting protein or peptide), wherein upon administration the tumor-targeting bacterium is specifically targeted to the tumor site. In some embodiments, the tumor-targeting bacterium comprises at least about 100 (e.g., at least about any of 150, 200, 250, 300, 400, or 500) copies of the tumor-targeting agent per bacterium. In some embodiments, the tumor-targeting agent is expressed by the tumor-targeting bacterium. In some embodiments, the tumor-targeting agent is attached to the surface of the tumor-targeting bacterium.

In another aspect, there is provided a method of delivering a therapeutic or diagnostic agent to a tumor site in an individual having cancer, comprising orally administering to the individual an effective amount of a tumor-targeting bacterium (e.g., Salmonella, such as Salmonella typhimurium, or Listeria, such as Listeria monocytogenes), wherein the tumor-targeting bacterium comprises on its surface a tumor-targeting agent (such as tumor-targeting protein or peptide), wherein the tumor-targeting bacterium further comprises the therapeutic or diagnostic agent, or a nucleic acid encoding the therapeutic or diagnostic agent, wherein upon administration the tumor-targeting bacterium is specifically targeted to the tumor site, thereby delivering the therapeutic or diagnostic agent to the tumor site. In some embodiments, the tumor-targeting bacterium comprises at least about 100 (e.g., at least about any of 150, 200, 250, 300, 400, or 500) copies of the tumor-targeting agent per bacterium. In some embodiments, the tumor-targeting agent is expressed by the tumor-targeting bacterium. In some embodiments, the tumor-targeting agent is attached to the surface of the tumor-targeting bacterium. In some embodiments, the therapeutic or diagnostic agent (or nucleic acid encoding the therapeutic or diagnostic agent) is loaded in the tumor-targeting bacteria. In some embodiments, the therapeutic or diagnostic agent (or nucleic acid encoding the therapeutic or diagnostic agent) is attached to the surface of the tumor-targeting bacterium. In some embodiments, the therapeutic or diagnostic agent is expressed by the tumor-targeting bacteria.

In some embodiments, there is provided a method of delivering a tumor-targeting bacterium (e.g., Salmonella, such as Salmonella typhimurium, or Listeria, such as Listeria monocytogenes) to a tumor site in an individual having cancer, comprising orally administering to the individual an effective amount of the tumor-targeting bacterium, wherein the tumor-targeting bacterium comprises on its surface a tumor-targeting peptide, wherein upon administration the tumor-targeting bacterium is specifically targeted to the tumor site. In some embodiments, the tumor-targeting bacterium comprises at least about 100 (e.g., at least about any of 150, 200, 250, 300, 400, or 500) copies of the tumor-targeting peptides per bacterium. In some embodiments, the tumor-targeting peptide is expressed by the tumor-targeting bacterium. In some embodiments, the tumor-targeting peptide is attached to the surface of the tumor-targeting bacterium.

In some embodiments, there is provided a method of delivering a therapeutic or diagnostic agent to a tumor site in an individual having cancer, comprising orally administering to the individual an effective amount of a tumor-targeting bacterium (e.g., Salmonella, such as Salmonella typhimurium, or Listeria, such as Listeria monocytogenes), wherein the tumor-targeting bacterium comprises on its surface a tumor-targeting peptide, wherein the tumor-targeting bacterium further comprises the therapeutic or diagnostic agent, or a nucleic acid encoding the therapeutic or diagnostic agent, wherein upon administration the tumor-targeting bacterium is specifically targeted to the tumor site, thereby delivering the therapeutic or diagnostic agent to the tumor site. In some embodiments, the tumor-targeting bacterium comprises at least about 100 (e.g., at least about any of 150, 200, 250, 300, 400, or 500) copies of the tumor-targeting peptides per bacterium. In some embodiments, the tumor-targeting peptide is expressed by the tumor-targeting bacterium. In some embodiments, the tumor-targeting peptide is attached to the surface of the tumor-targeting bacterium. In some embodiments, the tumor-targeting peptide is no longer than 25 amino acids in length. In some embodiments, the therapeutic or diagnostic agent (or nucleic acid encoding the therapeutic or diagnostic agent) is loaded in the tumor-targeting bacteria. In some embodiments, the therapeutic or diagnostic agent (or nucleic acid encoding the therapeutic or diagnostic agent) is attached to the surface of the tumor-targeting bacterium. In some embodiments, the therapeutic or diagnostic agent is expressed by the tumor-targeting bacteria.

In some embodiments, there is provided a method of delivering a tumor-targeting bacterium (e.g., Salmonella, such as Salmonella typhimurium, or Listeria, such as Listeria monocytogenes) to a tumor site in an individual having cancer, comprising orally administering to the individual an effective amount of the tumor-targeting bacterium, wherein the tumor-targeting bacterium comprises on its surface a PLZ peptide, wherein upon administration the tumor-targeting bacterium is specifically targeted to the tumor site. In some embodiments, the tumor-targeting bacterium comprises at least about 100 (e.g., at least about any of 150, 200, 250, 300, 400, or 500) copies of the PLZ peptides per bacterium. In some embodiments, the PLZ peptide is expressed by the tumor-targeting bacterium. In some embodiments, the PLZ peptide is attached to the surface of the tumor-targeting bacterium.

In some embodiments, there is provided a method of delivering a therapeutic or diagnostic agent to a tumor site in an individual having cancer, comprising orally administering to the individual an effective amount of a tumor-targeting bacterium (e.g., Salmonella, such as Salmonella typhimurium, or Listeria, such as Listeria monocytogenes), wherein the tumor-targeting bacterium comprises on its surface a PLZ peptide, wherein the tumor-targeting bacterium further comprises the therapeutic or diagnostic agent, or a nucleic acid encoding the therapeutic or diagnostic agent, and wherein upon administration the tumor-targeting bacterium is specifically targeted to the tumor site, thereby delivering the therapeutic or diagnostic agent to the tumor site. In some embodiments, the tumor-targeting bacterium comprises at least about 100 (e.g., at least about any of 150, 200, 250, 300, 400, or 500) copies of the PLZ peptides per bacterium. In some embodiments, the PLZ peptide is expressed by the tumor-targeting bacteria. In some embodiments, the PLZ peptide is attached to the surface of the tumor-targeting bacteria. In some embodiments, the PLZ peptide is no longer than 25 amino acids in length. In some embodiments, the therapeutic or diagnostic agent (or nucleic acid encoding the therapeutic or diagnostic agent) is loaded in the tumor-targeting bacteria. In some embodiments, the therapeutic or diagnostic agent (or nucleic acid encoding the therapeutic or diagnostic agent) is attached to the surface of the tumor-targeting bacterium. In some embodiments, the therapeutic or diagnostic agent is expressed by the tumor-targeting bacteria.

In some embodiments, the PLZ peptide comprises the amino acid sequence X₁DGRX₅GF (SEQ ID NO:1), wherein X₁ is selected from the group consisting of Gln and Gly and X₅ is any amino acid other than cysteine, optionally wherein the X₅ is Met, Lys, Gly, Ala or Gly-Gly. In some embodiments, the tumor-targeting peptide comprises an amino acid sequence selected from the group consisting of QDGRMGF (SEQ ID NO:2), QDGRKGF (SEQ ID NO:3) and QDGRKGGF (SEQ ID NO:4).

In some embodiments, the PLZ peptide comprises CX₁DGRX₅GFC (SEQ ID NO: 5), wherein X₁ is selected from the group consisting of Gln and Gly and X₅ is any amino acid other than cysteine, optionally wherein the X₅ is Met, Lys, Gly, Ala or Gly-Gly. In some embodiments, the PLZ peptide comprises a PLZ4 peptide having amino acid sequence of CQDGRMGFC (SEQ ID NO:6). In some embodiments, the C is a D-cysteine residue. In some embodiments, the C is an L-cysteine residue.

In some embodiments, there is provided a method of delivering a tumor-targeting bacterium (e.g., Salmonella, such as Salmonella typhimurium, or Listeria, such as Listeria monocytogenes) to a tumor site in an individual having cancer, comprising orally administering to the individual an effective amount of the tumor-targeting bacterium, wherein the tumor-targeting bacterium comprises on its surface a luteinizing hormone releasing hormone (LHRH) peptide, wherein upon administration the tumor-targeting bacterium is specifically targeted to the tumor site. In some embodiments, the tumor-targeting bacterium comprises at least about 100 (e.g., at least about any of 150, 200, 250, 300, 400, or 500) copies of the LHRH peptides per bacterium. In some embodiments, the LHRH peptide is expressed by the tumor-targeting bacteria. In some embodiments, the LHRH peptide is attached to the surface of the tumor-targeting bacteria. In some embodiments, the LHRH peptide is no longer than 25 amino acids in length. In some embodiments, the LHRH peptide comprises a peptide having the amino acid sequence of QHWSYGLRP (SEQ ID NO:7).

In some embodiments, there is provided a method of delivering a therapeutic or diagnostic agent to a tumor site in an individual having cancer, comprising orally administering to the individual an effective amount of a tumor-targeting bacterium (e.g., Salmonella, such as Salmonella typhimurium, or Listeria, such as Listeria monocytogenes), wherein the tumor-targeting bacterium comprises on its surface a LHRH peptide, wherein the tumor-targeting bacterium further comprises the therapeutic or diagnostic agent, or a nucleic acid encoding the therapeutic or diagnostic agent, and wherein upon administration the tumor-targeting bacterium is specifically targeted to the tumor site, thereby delivering the therapeutic or diagnostic agent to the tumor site. In some embodiments, the tumor-targeting bacterium comprises at least about 100 (e.g., at least about any of 150, 200, 250, 300, 400, or 500) copies of the LHRH peptides per bacterium. In some embodiments, the LHRH peptide is expressed by the tumor-targeting bacteria. In some embodiments, the LHRH peptide is attached to the surface of the tumor-targeting bacteria. In some embodiments, the LHRH peptide is no longer than 25 amino acids in length. In some embodiments, the therapeutic or diagnostic agent (or nucleic acid encoding the therapeutic or diagnostic agent) is loaded in the tumor-targeting bacteria. In some embodiments, the therapeutic or diagnostic agent (or nucleic acid encoding the therapeutic or diagnostic agent) is attached to the surface of the tumor-targeting bacterium. In some embodiments, the therapeutic or diagnostic agent is expressed by the tumor-targeting bacteria. In some embodiments, the LHRH peptide comprises a peptide having the amino acid sequence of QHWSYGLRP (SEQ ID NO:7).

The tumor-targeting peptide described herein can be embedded in a bacterial protein presented at the outer wall of the tumor-targeting bacterium. For example, the tumor-targeting peptide can be fused to the N- or C-terminus of a bacterial protein presented at the outer wall of the tumor-targeting bacterium, or inserted in the middle of the bacterial protein. In some embodiments when the bacteria is Salmonella or Listeria, the tumor-targeting peptide can be embedded in the outer protein A (OmpA), for example inserted at the third loop of OmpA.

The methods described herein can be useful for treating cancer. Thus, for example, in some embodiments, there is provided a method of treating an individual having cancer, comprising delivering a tumor-targeting bacterium (e.g., Salmonella, such as Salmonella typhimurium, or Listeria, such as Listeria monocytogenes) to the tumor site of the individual, wherein the tumor-targeting bacterium is delivered by orally administering to the individual an effective amount of the tumor-targeting bacterium, wherein the tumor-targeting bacterium comprises on its surface a tumor-targeting agent. In some embodiments, there is provided a method of treating an individual having cancer, comprising delivering a tumor-targeting bacterium (e.g., Salmonella, such as Salmonella typhimurium, or Listeria, such as Listeria monocytogenes) to the tumor site of the individual, wherein the tumor-targeting bacterium is delivered by orally administering to the individual an effective amount of the tumor-targeting bacterium, wherein the tumor-targeting bacterium comprises on its surface a tumor-targeting agent, and wherein the tumor-targeting bacterium further comprises a therapeutic agent or a nucleic acid encoding the therapeutic agent or a diagnostic or therapeutic agent loaded in or on the tumor-targeting bacterium. In some embodiments, the tumor-targeting agent is a tumor-targeting peptide, such as a PLZ peptide or a LHRH peptide, including any of the peptides described herein. The tumor-targeting agent (such as the tumor-targeting peptide) can be expressed and presented on the surface of the tumor-targeting bacteria, or it can be attached to the surface of the tumor-targeting bacteria. In some embodiments, the tumor-targeting bacterium is administered at about 1×10⁷ to 1×10¹³ Colony forming units (CFU) per dose, including for example any of 5×10⁷, 1×10⁸, 5×10⁸, 1×10⁹, 5×10⁹, 1×10¹⁰, 5×10¹⁰, 1×10¹¹, 5×10¹¹, 1×10¹², or 5×10¹² CFU per dose. In some embodiments, the tumor-targeting bacterium is administered at least about 1, 2, 3, 4, or 5 times daily. In some embodiments, the tumor-targeting bacterium is administered 1, 2, 3, 4, 5, 6, or 7 times weekly. In some embodiments, the tumor-targeting bacterium is administered for at least a year. In some embodiments, the tumor-targeting bacterium is administered with an empty stomach. In some embodiments, the tumor-targeting bacterium is administered immediately after a meal. In some embodiments, the individual has a cancer selected from the group consisting of breast cancer, prostate cancer, and bladder cancer. In some embodiments, the individual has a healthy gut microbiome. In some embodiments, the individual is a human individual.

In some embodiments, there is provided a method of treating an individual having cancer, comprising delivering a tumor-targeting bacterium (e.g., Salmonella, such as Salmonella typhimurium, or Listeria, such as Listeria monocytogenes) to the tumor site of the individual, wherein the tumor-targeting bacterium is delivered by orally administering to the individual an effective amount of the tumor-targeting bacterium, wherein the tumor-targeting bacterium comprises on its surface a tumor-targeting agent, and wherein the tumor-targeting bacterium further comprises a therapeutic agent or a nucleic acid encoding the therapeutic agent or a diagnostic or therapeutic agent loaded in or on the tumor-targeting bacterium. In some embodiments, the tumor-targeting agent is a tumor-targeting peptide, such as a PLZ peptide or a LHRH peptide, including any of the peptides described herein. The tumor-targeting agent (such as the tumor-targeting peptide) can be expressed and presented on the surface of the tumor-targeting bacteria, or it can be attached to the surface of the tumor-targeting bacteria. In some embodiments, the therapeutic agent is selected from the group consisting of: a protein (such as chemokine e.g., CXCL11), an antibody, an mRNA, an siRNA, an antisense RNA, and a CRISPR/gRNA system. In some embodiments, the therapeutic agent is an immune check point inhibitor, optionally selected from the group consisting of PD1-Fc, PD-L1-Fc, anti-PD1 antibody, and an anti-PD-L1 antibody. In some embodiments, the therapeutic agent is a toxin or a photosensitizer. In some embodiments, the tumor-targeting bacterium is administered at about 1×10⁷ to 1×10¹³ Colony forming units (CFU) per dose, including for example any of 5×10⁷, 1×10⁸, 5×10⁸, 1×10⁹, 5×10⁹, 1×10¹⁰, 5×10¹⁰, 1×10¹¹, 5×10¹¹, 1×10¹², or 5×10¹² CFU per dose. In some embodiments, the tumor-targeting bacterium is administered at least about 1, 2, 3, 4, or 5 times daily. In some embodiments, the tumor-targeting bacterium is administered 1, 2, 3, 4, 5, 6, or 7 times weekly. In some embodiments, the tumor-targeting bacterium is administered for at least a year. In some embodiments, the tumor-targeting bacterium is administered with an empty stomach. In some embodiments, the tumor-targeting bacterium is administered immediately after a meal. In some embodiments, the individual has a cancer selected from the group consisting of breast cancer, prostate cancer, and bladder cancer. In some embodiments, the individual has a healthy gut microbiome. In some embodiments, the individual is a human individual.

In some embodiments according to any one of the methods described above, the method comprises orally administering two or more tumor-targeting bacteria. The two or more tumor-targeting bacteria can be administered simultaneously or separately. In some embodiments, at least two of the two or more tumor-targeting bacteria comprise a different bacteria. In some embodiments, at least two of the two or more tumor-targeting bacteria comprise a different tumor-targeting agent. In some embodiments, at least two of the two or more tumor-targeting bacteria comprise a different bacteria and a different tumor-targeting agent. In some embodiments where the tumor-targeting bacteria comprise a therapeutic or diagnostic agent, at least two of the two or more tumor-targeting bacteria comprise a different therapeutic or diagnostic agent.

In some embodiments according to any one of the methods described above, the method further comprises orally administering a bacterium which is not a tumor-targeting bacteria (hereinafter referred to as “non-targeting bacterium”). The non-targeting bacterium can be administered separately or simultaneously with the tumor-targeting bacteria.

### Oral Formulations of the Present Invention

The present application in another aspect provides oral formulations of tumor-targeting bacteria. Such oral formulations of tumor-targeting bacteria can be administered to an individual as cancer therapeutics themselves, or be used as a delivery vehicle to delivery therapeutic or diagnostic agents to a tumor site.

In some embodiments, there is provided an oral formulation of a tumor-targeting bacterium (e.g., Salmonella, such as Salmonella typhimurium, or Listeria, such as Listeria monocytogenes) comprising on its surface a tumor-targeting agent and a pharmaceutically acceptable excipient. In some embodiments, the tumor-targeting bacterium comprises at least about 100 (e.g., at least about any of 150, 200, 250, 300, 400, or 500) copies of the tumor-targeting agent per bacterium. In some embodiments, the tumor-targeting agent is expressed by the tumor-targeting bacterium. In some embodiments, the tumor-targeting agent is attached to the surface of the tumor-targeting bacterium. In some embodiments, the oral formulation comprises one or more other bacteria. In some embodiments, the one or more other bacteria comprises tumor-targeting bacteria. In some embodiments, the one or more bacteria comprises bacteria which are not tumor-targeting bacteria.

In some embodiments, there is provided an oral formulation of a tumor-targeting bacterium (e.g., Salmonella, such as Salmonella typhimurium, or Listeria, such as Listeria monocytogenes) comprising on its surface a tumor-targeting agent and a pharmaceutically acceptable excipient, wherein the tumor-targeting bacterium further comprises a therapeutic or diagnostic agent or a nucleic acid encoding a therapeutic or diagnostic agent. In some embodiments, the tumor-targeting bacterium comprises at least about 100 (e.g., at least about any of 150, 200, 250, 300, 400, or 500) copies of the tumor-targeting agent per bacterium. In some embodiments, the tumor-targeting agent is expressed by the tumor-targeting bacterium. In some embodiments, the tumor-targeting agent is attached to the surface of the tumor-targeting bacterium. In some embodiments, the tumor-targeting bacterium comprises at least about 100 (e.g., at least about any of 150, 200, 250, 300, 400, or 500) copies of the tumor-targeting agent per bacterium. In some embodiments, the tumor-targeting agent is expressed by the tumor-targeting bacterium. In some embodiments, the tumor-targeting agent is attached to the surface of the tumor-targeting bacterium. In some embodiments, the therapeutic or diagnostic agent (or nucleic acid encoding the therapeutic or diagnostic agent) is loaded in the tumor-targeting bacteria. In some embodiments, the therapeutic or diagnostic agent (or nucleic acid encoding the therapeutic or diagnostic agent) is attached to the surface of the tumor-targeting bacterium. In some embodiments, the therapeutic or diagnostic agent is expressed by the tumor-targeting bacteria. In some embodiments, the oral formulation comprises one or more other bacteria. In some embodiments, the one or more other bacteria comprises tumor-targeting bacteria. In some embodiments, the one or more bacteria comprises bacteria which are not tumor-targeting bacteria.

In some embodiments, there is provided an oral formulation of a tumor-targeting bacterium (e.g., Salmonella, such as Salmonella typhimurium, or Listeria, such as Listeria monocytogenes) comprising on its surface a tumor-targeting peptide and a pharmaceutically acceptable excipient. In some embodiments, the tumor-targeting bacterium comprises at least about 100 (e.g., at least about any of 150, 200, 250, 300, 400, or 500) copies of the tumor-targeting peptide per bacterium. In some embodiments, the tumor-targeting peptide is expressed by the tumor-targeting bacterium. In some embodiments, the tumor-targeting peptide is attached to the surface of the tumor-targeting bacterium. In some embodiments, the oral formulation comprises one or more other bacteria. In some embodiments, the one or more other bacteria comprises tumor-targeting bacteria. In some embodiments, the one or more bacteria comprises bacteria which are not tumor-targeting bacteria.

In some embodiments, there is provided an oral formulation of a tumor-targeting bacterium (e.g., Salmonella, such as Salmonella typhimurium, or Listeria, such as Listeria monocytogenes) comprising on its surface a tumor-targeting peptide and a pharmaceutically acceptable excipient, wherein the tumor-targeting bacterium further comprises a therapeutic or diagnostic agent or a nucleic acid encoding a therapeutic or diagnostic agent. In some embodiments, the tumor-targeting bacterium comprises at least about 100 (e.g., at least about any of 150, 200, 250, 300, 400, or 500) copies of the tumor-targeting peptide per bacterium. In some embodiments, the tumor-targeting peptide is expressed by the tumor-targeting bacterium. In some embodiments, the tumor-targeting peptide is attached to the surface of the tumor-targeting bacterium. In some embodiments, the therapeutic or diagnostic agent (or nucleic acid encoding the therapeutic or diagnostic agent) is loaded in the tumor-targeting bacteria. In some embodiments, the therapeutic or diagnostic agent (or nucleic acid encoding the therapeutic or diagnostic agent) is attached to the surface of the tumor-targeting bacterium. In some embodiments, the therapeutic or diagnostic agent is expressed by the tumor-targeting bacteria. In some embodiments, the oral formulation comprises one or more other bacteria. In some embodiments, the one or more other bacteria comprises tumor-targeting bacteria. In some embodiments, the one or more bacteria comprises bacteria which are not tumor-targeting bacteria.

In some embodiments, there is provided an oral formulation of a tumor-targeting bacterium (e.g., Salmonella, such as Salmonella typhimurium, or Listeria, such as Listeria monocytogenes) comprising on its surface a PLZ peptide and a pharmaceutically acceptable excipient. In some embodiments, the tumor-targeting bacterium comprises at least about 100 (e.g., at least about any of 150, 200, 250, 300, 400, or 500) copies of the PLZ peptide per bacterium. In some embodiments, the PLZ peptide is expressed by the tumor-targeting bacterium. In some embodiments, the PLZ peptide is attached to the surface of the tumor-targeting bacterium. In some embodiments, the oral formulation comprises one or more other bacteria. In some embodiments, the one or more other bacteria comprises tumor-targeting bacteria. In some embodiments, the one or more bacteria comprises bacteria which are not tumor-targeting bacteria.

In some embodiments, there is provided an oral formulation of a tumor-targeting bacterium (e.g., Salmonella, such as Salmonella typhimurium, or Listeria, such as Listeria monocytogenes) comprising on its surface a PLZ peptide and a pharmaceutically acceptable excipient, wherein the tumor-targeting bacterium further comprises a therapeutic or diagnostic agent or a nucleic acid encoding a therapeutic or diagnostic agent. In some embodiments, the tumor-targeting bacterium comprises at least about 100 (e.g., at least about any of 150, 200, 250, 300, 400, or 500) copies of the PLZ peptide per bacterium. In some embodiments, the PLZ peptide is expressed by the tumor-targeting bacterium. In some embodiments, the PLZ peptide is attached to the surface of the tumor-targeting bacterium. In some embodiments, the therapeutic or diagnostic agent (or nucleic acid encoding the therapeutic or diagnostic agent) is loaded in the tumor-targeting bacteria. In some embodiments, the therapeutic or diagnostic agent (or nucleic acid encoding the therapeutic or diagnostic agent) is attached to the surface of the tumor-targeting bacterium. In some embodiments, the therapeutic or diagnostic agent is expressed by the tumor-targeting bacteria. In some embodiments, the oral formulation comprises one or more other bacteria. In some embodiments, the one or more other bacteria comprises tumor-targeting bacteria. In some embodiments, the one or more bacteria comprises bacteria which are not tumor-targeting bacteria.

In some embodiments, the PLZ peptide comprises the amino acid sequence X₁DGRX₅GF (SEQ ID NO:1), wherein X₁ is selected from the group consisting of Gln and Gly and X₅ is any amino acid other than cysteine, optionally wherein the X₅ is Met, Lys, Gly, Ala or Gly-Gly. In some embodiments, the tumor-targeting peptide comprises an amino acid sequence selected from the group consisting of QDGRMGF (SEQ ID NO:2), QDGRKGF (SEQ ID NO:3) and QDGRKGGF (SEQ ID NO:4).

In some embodiments, the PLZ peptide comprises CX₁DGRX₅GFC (SEQ ID NO: 5), wherein X₁ is selected from the group consisting of Gln and Gly and X₅ is any amino acid other than cysteine, optionally wherein the X₅ is Met, Lys, Gly, Ala or Gly-Gly. In some embodiments, the PLZ peptide comprises a PLZ4 peptide having amino acid sequence of CQDGRMGFC (SEQ ID NO:6). In some embodiments, the C is a D-cysteine residue. In some embodiments, the C is an L-cysteine residue.

In some embodiments, there is provided an oral formulation of a tumor-targeting bacterium (e.g., Salmonella, such as Salmonella typhimurium, or Listeria, such as Listeria monocytogenes) comprising on its surface a LHRH peptide and a pharmaceutically acceptable excipient. In some embodiments, the tumor-targeting bacterium comprises at least about 100 (e.g., at least about any of 150, 200, 250, 300, 400, or 500) copies of the LHRH peptide per bacterium. In some embodiments, the LHRH peptide is expressed by the tumor-targeting bacterium. In some embodiments, the LHRH peptide is attached to the surface of the tumor-targeting bacterium. In some embodiments, the oral formulation comprises one or more other bacteria. In some embodiments, the one or more other bacteria comprises tumor-targeting bacteria. In some embodiments, the one or more bacteria comprises bacteria which are not tumor-targeting bacteria.

In some embodiments, there is provided an oral formulation of a tumor-targeting bacterium (e.g., Salmonella, such as Salmonella typhimurium, or Listeria, such as Listeria monocytogenes) comprising on its surface a LHRH peptide and a pharmaceutically acceptable excipient, wherein the tumor-targeting bacterium further comprises a therapeutic or diagnostic agent or a nucleic acid encoding a therapeutic or diagnostic agent. In some embodiments, the tumor-targeting bacterium comprises at least about 100 (e.g., at least about any of 150, 200, 250, 300, 400, or 500) copies of the LHRH peptide per bacterium. In some embodiments, the LHRH peptide is expressed by the tumor-targeting bacterium. In some embodiments, the LHRH peptide is attached to the surface of the tumor-targeting bacterium. In some embodiments, the therapeutic or diagnostic agent (or nucleic acid encoding the therapeutic or diagnostic agent) is loaded in the tumor-targeting bacteria. In some embodiments, the therapeutic or diagnostic agent (or nucleic acid encoding the therapeutic or diagnostic agent) is attached to the surface of the tumor-targeting bacterium. In some embodiments, the therapeutic or diagnostic agent is expressed by the tumor-targeting bacteria. In some embodiments, the LHRH peptide comprises a peptide having the amino acid sequence of QHWSYGLRP (SEQ ID NO:7). In some embodiments, the oral formulation comprises one or more other bacteria. In some embodiments, the one or more other bacteria comprises tumor-targeting bacteria. In some embodiments, the one or more bacteria comprises bacteria which are not tumor-targeting bacteria.

### Tumor-Targeting Bacteria

The tumor-targeting bacterium described herein can be any bacterium comprising a tumor-targeting agent described herein. In some embodiments, the tumor-targeting bacterium is gram-positive. In some embodiments, the tumor-targeting bacterium is gram-negative. In some embodiments, the bacterium where the tumor-targeting bacterium is derived from is a bacterium found in the gut microbiota.

In some embodiments, the tumor-targeting bacterium is an enteric bacterium. In some embodiments, the tumor-targeting bacterium is a gram-negative bacteria. In some embodiments, the tumor-targeting bacterium is a member of the Enterobacteriaceae family of bacteria. In some embodiments, the tumor-targeting bacterium is a member of the genus Salmonella. In some embodiments, the tumor-targeting bacterium is Salmonella typhimurium.

In some embodiments, the tumor-targeting bacterium is a gram-positive bacteria. In some embodiments, the tumor-targeting bacterium is a member of the genus Listeria. In some embodiments, the tumor-targeting bacterium is Listeria monocytogenes.

### Tumor-Targeting Agents

The tumor-targeting agents described herein can be any targeting moiety capable of targeting the tumor-targeting bacterium to a tumor site. In some embodiments, the tumor-targeting agent is a tumor-targeting peptide. In some embodiments, the tumor-targeting agent is a tumor-targeting protein.

In some embodiments, the tumor-targeting agent (such as tumor-targeting peptide or protein) specifically targets the bacterium to a site of bladder cancer. In some embodiments, the tumor-targeting agent (such as tumor-targeting peptide or protein) specifically targets the bacterium to a site of breast cancer. In some embodiments, the tumor-targeting agent (such as tumor-targeting peptide or protein) specifically targets the bacterium to a site of prostate cancer.

In some embodiments, the tumor-targeting bacterium is genetically engineered to express a tumor-targeting agent. In some embodiments, the tumor-targeting agent is expressed on the surface of the tumor-targeting bacterium. In some embodiments, the tumor-targeting bacterium expresses a tumor-targeting peptide on its surface. In some embodiments, the tumor-targeting bacterium expresses a tumor-targeting protein on its surface.

In some embodiments, the tumor-targeting agent is a tumor-targeting peptide. In some embodiments, the tumor-targeting peptide is 5 to 40 amino acids in length. In some embodiments, the tumor-targeting peptide is 5-40, 5-35, 5-30, 5-25, 5-20, 5-15, or 5-10 amino acids in length. In some embodiments, the tumor-targeting peptide is no longer than 25 amino acids in length, such as no longer than 25, 20, 15, or 10 amino acids in length.

In some embodiments, the tumor-targeting peptide is a PLZ peptide or a luteinizing hormone releasing hormone (LHRH) peptide.

PLZ peptides are described, for example, in U.S. Pat. No. 8,946,379, which is specifically incorporated herein by reference. In some embodiments, the tumor-targeting bacterium comprises on its surface a tumor-targeting agent, wherein the tumor-targeting agent comprises a tumor-targeting peptide comprising the amino acid sequence X₁DGRX₅GF (SEQ ID NO:1). In some embodiments, X₁ and X₅ are any amino acid other than cysteine (e.g., A, D, E, F, G, H, I, K, L, M, N, P, Q, R, S, T, V, W, Y). In some embodiments, X₁ is selected from Gln and Gly. In some embodiments, X₅ is any amino acid other than cysteine (e.g., A, D, E, F, G, H, I, K, L, M, N, P, Q, R, S, T, V, W, Y). In some embodiments, X₅ is Met, Lys, Gly, Ala or Gly-Gly. In some embodiments, the tumor-targeting peptide comprises an amino acid sequence selected from the group consisting of QDGRMGF (SEQ ID NO:2), QDGRKGF (SEQ ID NO:3) and QDGRKGGF (SEQ ID NO:4).

In some embodiments, the tumor-targeting bacterium comprises on its surface a tumor-targeting agent, wherein the tumor-targeting agent comprises a tumor-targeting peptide comprising the amino acid sequence CX₁DGRX₅GFC (SEQ ID NO:5). In some embodiments, X₁ and X₅ are any amino acid other than cysteine (e.g., A, D, E, F, G, H, I, K, L, M, N, P, Q, R, S, T, V, W, Y). In some embodiments, X₁ is selected from Gln, Gly or Ala, and X₅ is any amino acid other than cysteine. In some embodiments, X₁ is any amino acid other than cysteine and X₅ is Met, Lys, Gly, Ala or Gly-Gly. In some embodiments, X₁ is selected from Gln, Gly or Ala, and X₅ is Met, Lys, Gly, Ala or Gly-Gly. In some embodiments, the tumor-targeting peptide comprises a PLZ4 peptide having amino acid sequence of CQDGRMGFC (SEQ ID NO:6). In some embodiments, the C is a D-cysteine residue. In some embodiments, the C is an L-cysteine residue.

In some embodiments, the tumor-targeting bacterium comprises on its surface a tumor-targeting agent, wherein the tumor-targeting agent comprises a luteinizing hormone releasing hormone (LHRH) peptide having the amino acid sequence of QHWSYGLRP (SEQ ID NO: 7).

In some embodiments, the tumor-targeting peptide is embedded in a bacterial protein presented at the outer wall of the tumor-targeting bacterium. In some embodiments, the outer wall is the outer membrane of a gram negative bacterium.

In some embodiments, the tumor-targeting peptide is fused to a bacterial signal peptide. In some embodiments, the signal peptide directs the tumor-targeting peptide to the outer surface of the bacterium.

In some embodiments, the tumor-targeting peptide is fused to an outer membrane protein. In some embodiments, the tumor-targeting peptide is fused to the N-terminus of the outer membrane protein. In some embodiments, the tumor-targeting peptide is fused to the C-terminus of the outer membrane protein. In some embodiments, the tumor-targeting peptide is inserted within the outer membrane protein. In some embodiments, the outer membrane protein is a beta-barrel protein. In some embodiments, the tumor-targeting peptide is inserted into an extracellular loop of the outer membrane protein. In some embodiments, the outer membrane protein is OmpA. In some embodiments, the tumor-targeting peptide is fused to the third loop of OmpA. Bacterial outer membrane proteins presenting the tumor-targeting peptide can be obtained, for example, by introducing into the bacterium a nucleic acid encoding such protein and allowing the protein to be presented on surface of the outer membrane.

In some embodiments, two or more tumor-targeting peptides are presented by an outer membrane protein. For example, an outer membrane protein can be modified by fusing 1, 2, 3, 4, or more tumor-targeting peptide to its N-terminus or C-terminus, and/or inserting 1, 2, 3, 4, or more tumor-targeting peptide within the ourter membrane protein.

In some embodiments, the tumor-targeting bacterium comprises on its surface a tumor-targeting agent, wherein the tumor-targeting agent comprises a tumor-targeting peptide comprising the amino acid sequence X₁DGRX₅GF (SEQ ID NO:1), wherein the tumor-targeting peptide is fused to an outer membrane protein. In some embodiments, the outer membrane protein is OmpA. In some embodiments, the tumor-targeting peptide comprising the amino acid sequence X₁DGRX₅GF (SEQ ID NO:1) is fused to an extracellular loop of OmpA. In some embodiments, the tumor-targeting peptide comprises an amino acid sequence selected from the group consisting of QDGRMGF (SEQ ID NO:2), QDGRKGF (SEQ ID NO: 3) and QDGRKGGF (SEQ ID NO:4). In some embodiments, the tumor-targeting peptide fused to OmpA comprises the amino acid sequence selected from the group consisting of SEQ ID NOs: 8-10.

In some embodiments, the tumor-targeting bacterium comprises on its surface a tumor-targeting agent, wherein the tumor-targeting agent comprises a tumor-targeting peptide comprising the amino acid sequence CX₁DGRX₅GFC (SEQ ID NO:5), wherein the tumor-targeting peptide is fused to an outer membrane protein. In some embodiments, the outer membrane protein is OmpA. In some embodiments, the tumor-targeting peptide comprising the amino acid sequence CX₁DGRX₅GFC (SEQ ID NO:5) is fused to an extracellular loop of OmpA. In some embodiments, the tumor-targeting peptide comprises PLZ4 peptide having amino acid sequence of CQDGRMGFC (SEQ ID NO:6). In some embodiments, the C is a D-cysteine residue. In some embodiments, the C is an L-cysteine residue. In some embodiments, the tumor-targeting peptide fused to OmpA comprises the amino acid sequence of SEQ ID NO: 11.

In some embodiments, the tumor-targeting bacterium comprises on its surface a tumor-targeting agent, wherein the tumor-targeting agent comprises a luteinizing hormone releasing hormone (LHRH) peptide having the amino acid sequence of QHWSYGLRP (SEQ ID NO: 7), wherein the tumor-targeting peptide is fused to an outer membrane protein. In some embodiments, the outer membrane protein is OmpA. In some embodiments, the tumor-targeting peptide fused to OmpA comprises the amino acid sequence of SEQ ID NO: 12.

In some embodiments, the tumor-targeting agent is a tumor-targeting protein. In some embodiments, the tumor-targeting protein is an antibody, such as a monoclonal antibody, a polyclonal antibody, a multispecific antibody (e.g., a bispecific antibody), a full-length antibody or an antigen-binding fragments thereof, so long as it exhibits the desired targeting activity.

In some embodiments, the tumor-targeting protein is an antibody fragment, such as a single-chain antibody (scFv). In some embodiments, the scFv is an anti-HER2 scFv. HER2 is also known as CD340, Neu, or ERBB2 and is a member of the epidermal growth factor receptor family.

In some embodiments, the tumor-targeting protein is a ligand or receptor, or portion thereof, such as the extracellular domain of a ligand/receptor. In some embodiments, the tumor-targeting protein is a ligand that binds a receptor on a cancer cell. In some embodiments, the tumor-targeting protein is a receptor that binds a ligand on a cancer cell.

In some embodiments, the tumor-targeting agent (such as tumor-targeting peptide or tumor-targeting protein) is expressed by the bacterium. A bacterium can be engineered to express the tumor-targeting peptide or protein by introducing into the bacterium a nucleic acid encoding the tumor-targeting peptide or protein (or a fusion protein comprising such tumor-targeting peptide or protein) into the bacterium, and allowing the expression and presentation of the tumor-targeting peptide or protein on the surface of the bacterium. In some embodiments, the tumor-targeting protein is fused to a bacterial signal peptide. In some embodiments, the signal peptide directs the tumor-targeting protein to the outer surface of the bacterium.

In some embodiments, the tumor-targeting agent (such as tumor-targeting peptide or tumor-targeting protein) is attached to the surface of the bacterium. Suitable methods for attaching an agent (such as a peptide or protein) to the surface of a bacterium are known in the art. See, e.g., Pham et al., Catalysts 2019, 9, 443; and Bi et al., Chemistry 2018 7; 24 (32:8042-8050, both of which are hereby incorporated by reference in their entirety.

In some embodiments, the tumor-targeting bacterium comprises at least about 50 copies of the tumor tumor-targeting agent per bacterium. In some embodiments, the tumor-targeting bacterium comprises at least about 100, at least about 150, at least about 200, at least about 250, at least about 500, at least about 1000, at least about 2500, or at least about 5000 copies of the tumor-targeting agent per bacterium.

In some embodiments, the tumor-targeting bacterium comprises about 50 to about 5000 copies of the tumor-targeting agent per bacterium. In some embodiments, the tumor-targeting bacterium comprises about 50 to about 100, about 100 to about 150, about 100 to about 200, about 100 to about 250, about 100 to about 500, about 100 to about 1000, about 100 to about 5000, about 150 to about 200, about 200 to about 250, about 250 to about 500, about 500 to about 1000, about 1000 to about 2500, or about 2500 to about 5000 copies of the tumor-targeting agent per bacterium.

### Therapeutic or Diagnostic Agents

In some embodiments, provided herein are methods of delivering a tumor-targeting bacterium to a tumor site in an individual having cancer, wherein the tumor-targeting bacterium comprises on its surface a tumor-targeting agent, wherein the tumor-targeting bacterium further comprises the therapeutic or diagnostic agent, or a nucleic acid encoding the therapeutic or diagnostic agent, or a therapeutic or diagnostic agent loaded in or on the tumor-targeting bacterium.

Any therapeutic or diagnostic agent that can be loaded into, expressed in, or exposed on the surface of a bacterium is suitable for the methods described herein.

In some embodiments, the therapeutic or diagnostic agent is a protein. In some embodiments, the protein is any one of an enzyme, an antibody or antibody fragment, a cytokine, a DNA editing protein (e.g., a targeted endonuclease such as a zinc finger nuclease, a transcription-activator-like nuclease (TALEN), a Cas9 protein, a Cas12 protein, or variants thereof), or an antibody or antibody fragment. In some embodiments, the therapeutic or diagnostic agent is an antibody or an antibody fragment.

In some embodiments, the therapeutic or diagnostic agent is a cytokine or a chemokine. In some embodiments, the therapeutic agent is CXCL11.

In some embodiments, the therapeutic or diagnostic agent is an immune check point inhibitor. In some embodiments, the immune checkpoint inhibitor inhibits any one of, but not limited to, A2AR, B7-H3, B7-H4, BTLA, CTLA-4, IDO, KIR, LAG3, NOX2, PD-1, PD-L1, PD-L2, TIM-3, VISTA, or SIGLEC7.

In some embodiments, the immune checkpoint inhibitor is an antibody that inhibits any one of, but not limited to, A2AR, B7-H3, B7-H4, BTLA, CTLA-4, IDO, KIR, LAG3, NOX2, PD-1, PD-L1, PD-L2, TIM-3, VISTA, or SIGLEC7. In some embodiments, the immune checkpoint inhibitor is any one of an PD-1-Fc, an PD-L1-Fc, an anti-PD-1 antibody, or an anti-PD-L1 antibody.

In some embodiments, the immune checkpoint inhibitor is an anti-PD-1 antibody. Exemplary anti-PD-1 antibodies include, but are not limited to, pembrolizumab (e.g., Keytruda®), cemiplimab (Libtayo®), and nivolumab (e.g., Opdivo®). In some embodiments, the antibody or antigen-binding domain specifically recognizing PD-1 comprises heavy chain CDRs, light chain CDRs, or all six CDRs of pembrolizumab, cemiplimab, or nivolumab.

In some embodiments, the immune checkpoint inhibitor is an anti-PD-L1 antibody. Exemplary anti-PDL1 antibodies include, but are not limited to Atezolizumab, Avelumab, Durvalumab, atezolizumab (e.g., Tecentriq®), avelumab (e.g., Bavencio®), and durvalumab (e.g., IMFINZI™).

In some embodiments, the therapeutic agent is a radioisotope.

In some embodiments, the therapeutic or diagnostic agent is a nucleic acid. In some embodiments, the nucleic acid is DNA, an mRNA, an siRNA, or an antisense RNA. In some embodiments, the nucleic acid encodes a protein.

In some embodiments, the therapeutic or diagnostic agent comprises one or more components of a CRISPR/gRNA system, e.g., a Cas protein and a gRNA.

In some embodiments, the therapeutic agent is for gene therapy. For example, the therapeutic agent can be a vector comprising a therapeutic gene, optionally under the control of a regulatory element (for example a promoter, such as an inducible promoter). In some embodiments, the therapeutic agent is a vector comprising a therapeutic gene that can correct one or more defective and/or mutated genes. In some embodiments, the therapeutic agent is a vector comprising a therapeutic gene that alters the function of one or more proteins. In some embodiments, the therapeutic agent is a vector comprising a therapeutic gene that alters the expression level of one or more genes. In some embodiments, the therapeutic agent is a vector that can change the sequence of one or more genes and/or regulator elements of the one more more genes. In some embodiments, the therapeutic agent is a vector comprising a therapeutic gene that is expressed at a target site, such as at a tumor site targeted by the tumor-targeting bacterium.

In some embodiments, the diagnostic agent is a detectable agent (such as a label, an enzyme, a radioactive agent, a fluorescent agent, for example a fluorescent protein).

In some embodiments, the tumor-targeting bacterium is engineered to contain or express the therapeutic or diagnostic agent (such as proteins, peptides, siRNAs, guide RNAs, etc.). The engineered bacterium can be obtained, for example, by introducing a nucleic acid encoding the therapeutic or diagnostic agent into the bacterium.

In some embodiments, the therapeutic or diagnostic agent is loaded into the tumor-targeting bacteria, for example, as described in Feng et al., Front. Chem. 2021; 9:689017, which is hereby incorporated by reference in its entirety.

In some embodiments, the therapeutic or diagnostic agent is attached to the surface of the bacteria, either directly or indirectly. Methods of attaching an agent to the surface of a bacterium is known in the art. See, e.g., Atwal et al., J. Microbiological Methods 130 (2016): 169-176.

### Dosing

Provided herein are methods of delivering a tumor-targeting bacterium to a tumor site in an individual having cancer, comprising orally administering to the individual an effective amount of the tumor-targeting bacterium or bacteria.

In some embodiments, the tumor-targeting bacterium is administered at a dose of about 5×10⁶ to about 10¹³ Colony forming units (CFU). In some embodiments, the tumor-targeting bacterium is administered at a dose of about 5×10⁶ to about 1×10⁷, about 1×10⁷ to about 5×10⁷, about 5×10⁷ to about 1×10⁸, about 1×10⁸ to about 5×10⁸, about 5×10⁸ to about 1×10⁹, about 1×10⁹ to about 5×10⁹, about 5×10⁹ to about 1×10¹⁰, about 1×10¹⁰ to about 5×10¹⁰, about 5×10¹⁰ to about 1×10¹¹, about 1×10¹¹ to about 5×10¹¹, about 5×10¹¹ to about 1×10¹², about 1×10¹² to about 5×10¹², about 5×10¹² to about 1×10¹³, about 5×10⁶ to about 5×10¹⁰, about 1×10⁷ to about 1×10¹⁰, about 1×10⁷ to about 5×10⁹, about 1×10⁷ to about 1×10⁸, about 1×10⁷ to about 5×10⁷, about 5×10⁹ to about 1×10¹⁰, about 1×10¹⁰ to about 5×10¹² CFU, or about 5×10¹⁰ to about 5×10¹² CFU. In some embodiments, the tumor-targeting bacterium is administered at a dose of about 1×10⁷, about 5×10⁷, about 1×10⁸, about 5×10⁸, about 1×10⁹, about 5×10⁹, about 1×10¹⁰, about 5×10¹⁰, about 1×10¹¹, about 5×10¹¹, about 1×10¹², or about 5×10¹² CFU.

In some embodiments, the tumor-targeting bacterium is administered for at least about 6 months to about 5 years. In some embodiments, the tumor-targeting bacterium is administered for about 6 months to about 1 year, about 1 year to about 1.5 years, about 1.5 years to about 2 years, about 2 years to about 3 years, about 3 years to about 4 years, about 4 years to about 5 years, or about 1 year to about 2 years. In some embodiments, the tumor-targeting bacterium is administered for about 6 months, about 7 months, about 8 months, about 9 months, about 10 months, about 11 months, about 1 year, about 2 years, about 3 years, about 4 years, about 5 years, or longer.

In some embodiments, the tumor-targeting bacterium is administered as a single dose or as multiple doses. In some embodiments, the tumor-targeting bacterium is administered once a day for 2 days, 3 days, 4 days, 5 days, 6 days, 1 week, 2 weeks 3 weeks, 1 month, 2 months, 3 months, 4 months, 5 months, 6 months, or 1 year. In some embodiments, the tumor-targeting bacterium is administered twice a day for 2 days, 3 days, 4 days, 5 days, 6 days, 1 week, 2 weeks 3 weeks, 1 month, 2 months, 3 months, 4 months, 5 months, 6 months, or 1 year. In some embodiments, the tumor-targeting bacterium is administered three times a day for 2 days, 3 days, 4 days, 5 days, 6 days, 1 week, 2 weeks 3 weeks, 1 month, 2 months, 3 months, 4 months, 5 months, 6 months, or 1 year. In some embodiments, the tumor-targeting bacterium is administered four times a day for 2 days, 3 days, 4 days, 5 days, 6 days, 1 week, 2 weeks 3 weeks, 1 month, 2 months, 3 months, 4 months, 5 months, 6 months, or 1 year. In some embodiments, the tumor-targeting bacterium is administered once a week for 2 weeks, 3 weeks, 4 weeks, 1 month, 2 months, 3 months, 4 months, 5 months, 6 months, or for at least 1 year. In some embodiments, the tumor-targeting bacterium is administered biweekly for 4 weeks, 1 month, 2 months, 3 months, 4 months, 5 months, 6 months, or for at least 1 year. In some embodiments, the tumor-targeting bacterium is administered once a month for 2 months, 3 months, 4 months, 5 months, 6 months, or for at least 1 year.

In some embodiments, the tumor-targeting bacterium is administered with food. In some embodiments, the tumor-targeting bacterium is administered immediately after a meal. In some embodiments, the tumor-targeting bacterium is administered with a liquid. In some embodiments, the tumor-targeting bacterium is administered with water. In some embodiments, the tumor-targeting bacterium is administered immediately before a meal. In some embodiments, the tumor-targeting bacterium is administered with empty stomach.

In some embodiments, the tumor-targeting bacterium is formulated into capsule form to prevent damage by digestive enzymes. In some embodiments, tumor-targeting bacterium is formulated an oral formulation such as any of the oral formulations described herein.

### Patient Populations

The methods provided herein comprise delivering a tumor-targeting bacterium to a tumor site in an individual having cancer through oral administration. In some embodiments, the individual has breast cancer, prostate cancer, or bladder cancer.

In some embodiments, the individual is a human individual. In some embodiments, the individual has been previously immunized with the bacterium from which the tumor-targeting bacterium is derived.

### EXEMPLARY EMBODIMENTS

In some embodiments, provided herein is a method of delivering a tumor-targeting bacterium to a tumor site in an individual having cancer, comprising orally administering to the individual an effective amount of the tumor-targeting bacterium or bacteria, wherein the tumor-targeting bacterium comprises on its surface a tumor-targeting agent, wherein upon administration the tumor-targeting bacterium is specifically targeted to the tumor site, wherein the individual has bladder cancer, and wherein the tumor-targeting agent comprises the amino acid sequence X₁DGRX₅GF (SEQ ID NO:1), wherein X₁ is selected from Gln and Gly and X₅ is any amino acid other than cysteine. In some embodiments, the bacterium is Salmonella. In some embodiments, the bacterium is Listeria. In some embodiments, the tumor-targeting agent comprises an amino acid sequence selected from the group consisting of QDGRMGF (SEQ ID NO:2), QDGRKGF (SEQ ID NO:3) and QDGRKGGF (SEQ ID NO: 4). In some embodiments, the tumor-targeting agent is fused to an outer membrane protein. In some embodiments, the outer membrane protein is OmpA. In some embodiments, the therapeutic or diagnostic agent is CXCL11 or haPD-1.

In some embodiments, provided herein is a method of delivering a therapeutic or diagnostic agent to a tumor site in an individual having cancer, comprising orally administering to the individual an effective amount of the tumor-targeting bacterium, wherein the tumor-targeting bacterium comprises on its surface a tumor-targeting agent, wherein the tumor-targeting bacterium further comprises the therapeutic or diagnostic agent, or a nucleic acid encoding the therapeutic or diagnostic agent, wherein upon administration the tumor-targeting bacterium is specifically targeted to the tumor site, thereby delivering the therapeutic or diagnostic agent to the tumor site, wherein the individual has bladder cancer, and wherein the tumor-targeting agent comprises the amino acid sequence X₁DGRX₅GF (SEQ ID NO:1), wherein X₁ is selected from Gln and Gly and X₅ is any amino acid other than cysteine. In some embodiments, the bacterium is Salmonella. In some embodiments, the bacterium is Listeria. In some embodiments, the tumor-targeting agent comprises an amino acid sequence selected from the group consisting of QDGRMGF (SEQ ID NO:2), QDGRKGF (SEQ ID NO:3) and QDGRKGGF (SEQ ID NO:4). In some embodiments, the tumor-targeting agent is fused to an outer membrane protein. In some embodiments, the outer membrane protein is OmpA. In some embodiments, the therapeutic or diagnostic agent is CXCL11 or haPD-1.

In some embodiments, provided herein is a method of delivering a tumor-targeting bacterium to a tumor site in an individual having cancer, comprising orally administering to the individual an effective amount of the tumor-targeting bacterium or bacteria, wherein the tumor-targeting bacterium comprises on its surface a tumor-targeting agent, wherein upon administration the tumor-targeting bacterium is specifically targeted to the tumor site, wherein the individual has bladder cancer, and wherein the tumor-targeting agent comprises the amino acid sequence CX₁DGRX₅GFC (SEQ ID NO:5), wherein X₁ is selected from Gln and Gly and X₅ is any amino acid other than cysteine. In some embodiments, X₅ is Met, Lys, Gly, Ala or Gly-Gly. In some embodiments, the bacterium is Salmonella. In some embodiments, the bacterium is Listeria. In some embodiments, the tumor-targeting agent comprises an amino acid sequence CQDGRMGFC (SEQ ID NO:6). In some embodiments, the C is a D-cysteine residue. In some embodiments, the C is an L-cysteine residue. In some embodiments, the tumor-targeting agent is fused to an outer membrane protein. In some embodiments, the outer membrane protein is OmpA. In some embodiments, the therapeutic or diagnostic agent is CXCL11 or haPD-1.

In some embodiments, provided herein is a method a therapeutic or diagnostic agent to a tumor site in an individual having cancer, comprising orally administering to the individual an effective amount of the tumor-targeting bacterium, wherein the tumor-targeting bacterium comprises on its surface a tumor-targeting agent, wherein the tumor-targeting bacterium further comprises the therapeutic or diagnostic agent, or a nucleic acid encoding the therapeutic or diagnostic agent, wherein upon administration the tumor-targeting bacterium is specifically targeted to the tumor site, thereby delivering the therapeutic or diagnostic agent to the tumor site, wherein the individual has bladder cancer, and wherein the tumor-targeting agent comprises the amino acid sequence CX₁DGRX₅GFC (SEQ ID NO:5), wherein X₁ is selected from Gln and Gly and X₅ is any amino acid other than cysteine. In some embodiments, X₅ is Met, Lys, Gly, Ala or Gly-Gly. In some embodiments, the bacterium is Salmonella. In some embodiments, the bacterium is Listeria. In some embodiments, the tumor-targeting agent comprises an amino acid sequence CQDGRMGFC (SEQ ID NO:6). In some embodiments, the C is a D-cysteine residue. In some embodiments, the C is an L-cysteine residue. In some embodiments, the tumor-targeting agent is fused to an outer membrane protein. In some embodiments, the outer membrane protein is OmpA. In some embodiments, the therapeutic or diagnostic agent is CXCL11 or haPD-1.

In some embodiments, provided herein is a method of delivering a tumor-targeting bacterium to a tumor site in an individual having cancer, comprising orally administering to the individual an effective amount of the tumor-targeting bacterium or bacteria, wherein the tumor-targeting bacterium comprises on its surface a tumor-targeting agent, wherein upon administration the tumor-targeting bacterium is specifically targeted to the tumor site, wherein the individual has breast cancer, and wherein the tumor-targeting agent is a luteinizing hormone-releasing hormone (LHRH). In some embodiments, the bacterium is Salmonella. In some embodiments, the bacterium is Listeria. In some embodiments, the LHRH has the amino acid sequence of QHWSYGLRP (SEQ ID NO:7). In some embodiments, the LHRH is fused to an outer membrane protein. In some embodiments, the outer membrane protein is OmpA. In some embodiments, the therapeutic or diagnostic agent is haPD-1.

In some embodiments, provided herein is a method a therapeutic or diagnostic agent to a tumor site in an individual having cancer, comprising orally administering to the individual an effective amount of the tumor-targeting bacterium, wherein the tumor-targeting bacterium comprises on its surface a tumor-targeting agent, wherein the tumor-targeting bacterium further comprises the therapeutic or diagnostic agent, or a nucleic acid encoding the therapeutic or diagnostic agent, wherein upon administration the tumor-targeting bacterium is specifically targeted to the tumor site, thereby delivering the therapeutic or diagnostic agent to the tumor site, wherein the individual has breast cancer, and wherein the tumor-targeting agent is a luteinizing hormone-releasing hormone (LHRH). In some embodiments, the bacterium is Salmonella. In some embodiments, the bacterium is Listeria. In some embodiments, the LHRH has the amino acid sequence of QHWSYGLRP (SEQ ID NO: 7). In some embodiments, the LHRH is fused to an outer membrane protein. In some embodiments, the outer membrane protein is OmpA. In some embodiments, the therapeutic or diagnostic agent is haPD-1.

### Oral Formulations

In some embodiments, provided herein is an oral formulation of tumor-targeting bacterium comprising on its surface a tumor-targeting agent and a pharmaceutically acceptable excipient. In some embodiments, the tumor-targeting bacterium further comprises a nucleic acid encoding a therapeutic agent.

Suitable formulations can be prepared by methods commonly employed using conventional, organic or inorganic additives, such as an excipient selected from fillers or diluents, binders, disintegrants, lubricants, flavoring agents, preservatives, stabilizers, suspending agents, dispersing agents, surfactants, antioxidants or solubilizers.

Excipients that may be selected are known to those skilled in the art and include, but are not limited to fillers or diluents (e.g., sucrose, starch, mannitol, sorbitol, lactose, glucose, cellulose, talc, calcium phosphate or calcium carbonate and the like), a binder (e.g., cellulose, carboxymethylcellulose, methylcellulose, hydroxymethylcellulose, hydroxypropylmethylcellulose, polypropylpyrrolidone, polyvinylpyrrolidone, gelatin, gum arabic, polyethyleneglycol or starch and the like), a disintegrants (e.g., sodium starch glycolate, croscarmellose sodium and the like), a lubricant (e.g., magnesium stearate, light anhydrous silicic acid, talc or sodium lauryl sulfate and the like), a flavoring agent (e.g., citric acid, or menthol and the like), a preservative (e.g., sodium benzoate, sodium bisulfite, methylparaben or propylparaben and the like), a stabilizer (e.g., citric acid, sodium citrate or acetic acid and the like), a suspending agent (e.g., methylcellulose, polyvinyl pyrrolidone or aluminum stearate and the like) a dispersing agent (e.g., hydroxypropylmethylcellulose and the like), surfactants (e.g., sodium lauryl sulfate, polaxamer, polysorbates and the like), antioxidants (e.g., ethylene diamine tetraacetic acid (EDTA), butylated hydroxyl toluene (BHT) and the like) and solubilizers (e.g., polyethylene glycols, polyoxyl 15 hydroxystearate (branded/marketed as SOLUTOL®), lauroyl polyoxylglycerides (branded/marketed as GELUCIRE®) and the like). The effective amount of the modified bacteria provided herein in the pharmaceutical composition may be at a level that will exercise the desired effect.

In some embodiments, provided herein are compositions comprising an effective amount of tumor-targeting bacteria provided herein and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

In some embodiments, the oral formulation is a lyophilized powder, capsule, tablet, troche, lozenge, granule, gel or liquid. In some embodiments, the oral formulation is tablet or pill and can be compressed, multiply compressed, multiply layered, and/or coated.

In some embodiments, the oral formulation comprises about 5×10⁶ to about 1×10⁷, about 1×10⁷ to about 5×10⁷, about 5×10⁷ to about 1×10⁸, about 1×10⁸ to about 5×10⁸, about 5×10⁸ to about 1×10⁹, about 1×10⁹ to about 5×10⁹, about 5×10⁹ to about 1×10¹⁰, about 1×10¹⁰ to about 5×10¹⁰, about 5×10¹⁰ to about 1×10¹¹, about 1×10¹¹ to about 5×10¹¹, about 5×10¹¹ to about 1×10¹², about 1×10¹² to about 5×10¹², about 5×10¹² to about 1×10¹³, about 5×10⁶ to about 5×10¹⁰, about 1×10⁷ to about 1×10¹⁰, about 1×10⁷ to about 5×10⁹, about 1×10⁷ to about 1×10⁸, about 1×10⁷ to about 5×10⁷, about 5×10⁹ to about 1×10¹⁰, about 1×10¹⁰ to about 5×10¹² CFU, or about 5×10¹⁰ to about 5×10¹² CFU CFU. In some embodiments, the tumor-targeting bacterium is administered at a dose of about 1×10⁷, about 5×10⁷, about 1×10⁸, about 5×10⁸, about 1×10⁹, about 5×10⁹, about 1×10¹⁰, about 5×10¹⁰, about 1×10¹¹, about 5×10¹¹, about 1×10¹², or about 5×10¹² CFU of the tumor-targeting bacterium.

### EXEMPLARY EMBODIMENTS

Embodiment 1. A method of delivering a tumor-targeting bacterium to a tumor site in an individual having cancer, comprising orally administering to the individual an effective amount of the tumor-targeting bacterium, wherein the tumor-targeting bacterium comprises on its surface a tumor-targeting agent, wherein upon administration the tumor-targeting bacterium is specifically targeted to the tumor site.

Embodiment 2. A method of delivering a therapeutic or diagnostic agent to a tumor site in an individual having cancer, comprising orally administering to the individual an effective amount of the tumor-targeting bacterium, wherein the tumor-targeting bacterium comprises on its surface a tumor-targeting agent, wherein the tumor-targeting bacterium further comprises the therapeutic or diagnostic agent or a nucleic acid encoding the therapeutic or diagnostic agent, wherein upon administration the tumor-targeting bacterium is specifically targeted to the tumor site, thereby delivering the therapeutic or diagnostic agent to the tumor site.

Embodiment 3. The method of embodiment 1 or 2, wherein the tumor-targeting agent is a tumor-targeting peptide or tumor-targeting protein.

Embodiment 4. The method of embodiment 3, wherein the tumor-targeting bacterium is genetically engineered to express the tumor-targeting peptide or tumor-targeting protein on its surface.

Embodiment 5. The method of any one of embodiments 1-3, wherein the tumor-targeting agent is attached to the surface of the tumor-targeting bacterium.

Embodiment 6. The method of any one of embodiments 3-5, wherein the tumor-targeting agent is a tumor-targeting peptide.

Embodiment 7. The method of embodiment 6, wherein the tumor-targeting peptide is no longer than 25 amino acids in length.

Embodiment 8. The method of embodiment 7, wherein the tumor-targeting peptide is selected from the group consisting of a PLZ peptide and a luteinizing hormone releasing hormone (LHRH) peptide.

Embodiment 9. The method of embodiment 6, wherein the tumor-targeting peptide comprises the amino acid sequence X₁DGRX₅GF (SEQ ID NO:1), wherein X₁ is selected from Gln and Gly and X₅ is any amino acid other than cysteine.

Embodiment 10. The method of embodiment 9, wherein the X₅ is Met, Lys, Gly, Ala or Gly-Gly.

Embodiment 11. The method of embodiment 10, wherein the tumor-targeting peptide comprises the amino acid sequence selected from the group consisting of QDGRMGF (SEQ ID NO:2), QDGRKGF (SEQ ID NO:3) and QDGRKGGF (SEQ ID NO: 4).

Embodiment 12. The method of embodiment 6, wherein the tumor-targeting peptide comprises the amino acid sequence CX₁DGRX₅GFC (SEQ ID NO:5), wherein X₁ is selected from Gln and Gly and X₅ is any amino acid other than cysteine.

Embodiment 13. The method of embodiment 12, wherein the X₅ is Met, Lys, Gly, Ala or Gly-Gly.

Embodiment 14. The method of embodiment 13, wherein the tumor-targeting peptide comprises PLZ4 peptide having an amino acid sequence of CQDGRMGFC (SEQ ID NO: 6).

Embodiment 15. The method of embodiment 6, wherein the tumor-targeting peptide comprises luteinizing hormone releasing hormone (LHRH) peptide having the amino acid sequence of QHWSYGLRP (SEQ ID NO:7).

Embodiment 16. The method of any one of embodiments 6-15, wherein the tumor-targeting peptide is embedded in a bacterial protein presented at the outer wall of the tumor-targeting bacterium.

Embodiment 17. The method of embodiment 16, wherein the tumor-targeting peptide is inserted at the third loop of salmonella outer protein A (OmpA).

Embodiment 18. The method of any one of embodiments 3-5, wherein the tumor-targeting agent is a tumor-targeting protein.

Embodiment 19. The method of any one of embodiments 1-18, wherein the tumor-targeting bacterium comprises at least about 100 copies of the tumor-targeting agent per bacterium.

Embodiment 20. The method of any one of embodiments 1-19, wherein the tumor-targeting bacterium is Salmonella.

Embodiment 21. The method of embodiment 20, wherein the tumor-targeting bacterium is Salmonella typhimurium.

Embodiment 22. The method of any one of embodiments 2-21, wherein the therapeutic or diagnostic agent is selected from the group consisting of: a protein (such as chemokine e.g., CXCL11), an antibody, an mRNA, an siRNA, an antisense RNA, and a CRISPR/gRNA system.

Embodiment 23. The method of embodiment 22, wherein the therapeutic agent is an immune check point inhibitor, optionally selected from the group consisting of PD1-Fc, PD-L1-Fc, anti-PD1 antibody, and an anti-PD-L1 antibody.

Embodiment 24. The method of embodiment 22, wherein the therapeutic agent is a toxin or a photosensitizer.

Embodiment 25. The method of any one of embodiments 1-24, wherein the tumor-targeting bacterium is administered at about 1×10⁷ to 5×10¹² Colony forming units (CFU) per dose.

Embodiment 26. The method of any one of embodiments 1-25, wherein the tumor-targeting bacterium is administered one to four times daily.

Embodiment 27. The method of any one of embodiments 1-26, wherein the tumor-targeting bacterium is administered for at least a year.

Embodiment 28. The method of any one of embodiments 1-27, wherein the tumor-targeting bacterium is administered with an empty stomach.

Embodiment 29. The method of any one of embodiments 1-27, wherein the tumor-targeting bacterium is administered immediately after a meal.

Embodiment 30. The method of any one of embodiments 1-29, wherein the individual has breast cancer, prostate cancer, or bladder cancer.

Embodiment 31. The method of any one of embodiments 1-30, wherein the individual has a healthy gut microbiome.

Embodiment 32. The method of any one of embodiments 1-31, wherein the individual is a human individual.

Embodiment 33. An oral formulation of tumor-targeting bacterium comprising on its surface a tumor-targeting agent and a pharmaceutically acceptable excipient.

Embodiment 34. The oral formulation of embodiment 33, wherein the tumor-targeting bacterium further comprises a therapeutic or diagnostic agent or a nucleic acid encoding a therapeutic or diagnostic agent.

Embodiment 35. The oral formulation of embodiments 33 and 34, wherein the oral formulation is in the form of capsule or liquid formulation.

Embodiment 36. The oral formulation of any one of embodiments 33-35, wherein the tumor-targeting agent is a tumor-targeting peptide or tumor-targeting protein.

Embodiment 37. The oral formulation of embodiment 36, wherein the tumor-targeting bacterium is genetically engineered to express the tumor-targeting peptide or tumor-targeting protein on its surface.

Embodiment 38. The oral formulation of any one of embodiments 34-37, wherein other targeting moiety is attached to the surface of the tumor-targeting bacterium.

Embodiment 39. The oral formulation of embodiment 36, wherein the tumor-targeting agent is a tumor-targeting peptide.

Embodiment 40. The oral formulation of embodiment 39, wherein the tumor-targeting peptide is no longer than 25 amino acids in length.

Embodiment 41. The oral formulation of embodiment 39-40, wherein the tumor-targeting peptide is selected from the group consisting of a PLZ peptide and a luteinizing hormone releasing hormone (LHRH) peptide.

Embodiment 42. The oral formulation of embodiment 41, wherein the tumor-targeting peptide comprises the amino acid sequence X₁DGRX₅GF (SEQ ID NO:1), wherein X₁ is selected from Gln and Gly and X₅ is any amino acid other than cysteine.

Embodiment 43. The oral formulation of embodiment 41, wherein the X₅ is Met, Lys, Gly, Ala or Gly-Gly.

Embodiment 44. The oral formulation of embodiment 41 or 42, wherein the tumor-targeting peptide comprises an amino acid sequence selected from the group consisting of QDGRMGF (SEQ ID NO:2), QDGRKGF (SEQ ID NO:3) and QDGRKGGF (SEQ ID NO: 4).

Embodiment 45. The oral formulation of embodiment 39, wherein the tumor-targeting peptide comprises CX₁DGRX₅GFC (SEQ ID NO:5), wherein X₁ is selected from Gln and Gly and X₅ is any amino acid other than cysteine.

Embodiment 46. The oral formulation of embodiment 45, wherein the X₅ is Met, Lys, Gly, Ala or Gly-Gly.

Embodiment 47. The oral formulation of embodiment 45, wherein the tumor-targeting peptide comprises PLZ4 peptide having amino acid sequence of CQDGRMGFC (SEQ ID NO:6).

Embodiment 48. The oral formulation of embodiment 39, wherein the tumor-targeting peptide comprises luteinizing hormone releasing hormone (LHRH) peptide having an amino acid sequence of QHWSYGLRP (SEQ ID NO:7).

Embodiment 49. The oral formulation of any one of embodiments 39-48, wherein the tumor-targeting peptide is embedded in a bacterial protein presented at the outer wall of the tumor-targeting bacterium.

Embodiment 50. The oral formulation of embodiment 49, wherein the tumor-targeting peptide is inserted at the third loop of salmonella outer protein A (OmpA).

Embodiment 51. The oral formulation of any one of embodiments 36-38, wherein the tumor-targeting agent is a tumor-targeting protein.

Embodiment 52. The oral formulation of any one of embodiments 33-51, wherein the tumor-targeting bacterium comprises at least about 100 copies of the tumor-targeting agent per bacterium.

Embodiment 53. The oral formulation of any one of embodiments 33-52, wherein the tumor-targeting bacterium is Salmonella.

Embodiment 54. The oral formulation of embodiment 53, wherein the tumor-targeting bacterium is Salmonella typhimurium.

Embodiment 55. The oral formulation of any one of embodiments 34-54, wherein the therapeutic or diagnostic agent is selected from the group consisting of: a protein (such as chemokine e.g., CXCL11), an antibody, an mRNA, an siRNA, an antisense RNA, and a CRISPR/gRNA system.

Embodiment 56. The oral formulation of embodiment 55, wherein the therapeutic agent is an immune check point inhibitor, optionally selected from the group consisting of PD1-Fc, PD-L1-Fc, anti-PD1 antibody, and an anti-PD-L1 antibody.

Embodiment 57. The oral formulation of any one of embodiments 34-54, wherein the therapeutic agent is a toxin or a photosensitizer.

Embodiment 58. The oral formulation of any one of embodiments 34-57, wherein the oral formulation comprises one or more other bacteria.

Embodiment 59. The oral formulation of embodiment 58, wherein the one or more other bacteria comprises tumor-targeting bacteria.

Embodiment 60. The oral formulation of embodiments 58 or 59, wherein the one or more bacteria comprises bacteria which are not tumor-targeting bacteria.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. The examples below are intended to be purely exemplary of the application and should therefore not be considered to limit the application in any way. The following examples and detailed description are offered by way of illustration and not by way of limitation.

### Example 1: Cancer Specific Delivery of Salmonella after Oral Administration

This example discusses the finding that a cancer-targeting peptide/protein expressed on the surface of bacteria Salmonella can guide cancer-specific delivery of Salmonella to cancer sites after oral administration. Since Salmonella can be used as a vector to deliver genes, cancer-targeting Salmonella can be used to specifically deliver genes of interest to cells/organs/cancers where Salmonella expresses the proteins of interest for diagnostic and therapeutic applications. In addition to cancer-targeting peptide/proteins, other tissue-targeting peptide/proteins can also be used to guide tissue-specific delivery of Salmonella for gene therapy or other applications.

When a cancer-targeting peptide/protein is expressed on the surface of Salmonella, that cancer-targeting peptide/protein can guide cancer-specific drug delivery to cancer sites after intravenous injection. Some Salmonella can target cancer based on the metabolic difference between cancer and normal cells. Approximately 100 trillion bacteria normally exist in the digestive system. Intestinal mucosa constitutes physical, biochemical and immune barriers that prevent bacteria inside the digestive system from entering into the body and going to other organs. Hence, bacteria in the gut usually do not enter into the blood circulation and cause disease. One study showed that oral bacteria could reach metastatic cancer liver, but only approximately 10⁶ colony-forming unit (CFU or live bacteria) per gram of tissue, or one bacterium per several hundreds of cancer cells (Danino et al. Sci Transl Med. 2015; 7: 289ra284).

A bladder cancer-targeting peptide named PLZ4 (the amino acid sequence: CQDGRMGFC, U.S. Pat. No. 8,946,379) was developed to specifically bind to bladder cancer cells, but not to normal cells, such as normal urothelial cells lining the cavity of the bladder, blood cells, fibroblasts, and human umbilical vascular endothelial cells. It can still bind to bladder cancer cells in pH6.0 urine. In this experiment, PLZ4 was expressed on the third loop of Salmonella Outer Membrane Protein A (OmpA) for cancer-specific targeting as previously reported (Park S H, Zheng J H, Nguyen V H, Jiang S N, Kim D Y, Szardenings M, Min J H, Hong Y, Choy H E, Min J J. RGD Peptide Cell-Surface Display Enhances the Targeting and Therapeutic Efficacy of Attenuated Salmonella-mediated Cancer Therapy. Theranostics. 2016 Jun. 20; 6 (10): 1672).

Cancer-Specific Delivery of PLZ4-Salmonella Expressing High-Affinity PD1-Enhanced Green Fluorescence Protein (haPD1/EGFP) Chimeric Protein

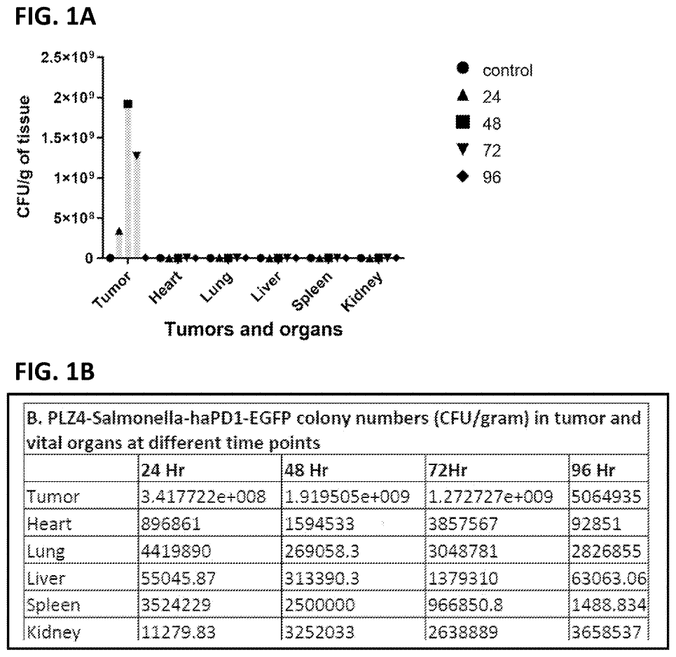

To determine whether PLZ4 can guide cancer-specific delivery of Salmonella, first, PLZ4-Salmonella, in which PLZ4 was expressed on the third loop of the Salmonella Outer Membrane Protein A (OmpA) on the surface of Salmonella, was constructed. This PLZ4-Salmonella construct expressed a chimeric protein high affinity Programmed Cell Death 1 (PD1) conjugated to enhanced green fluorescent protein (haPD1-EGFP). Mice carrying subcutaneous bladder cancer implants were used for this experiment. After oral administration of 5×10⁸ CFU (colony-forming unit, representing viable Salmonella) PLZ4-Salmonella-haPD1-EGFP per mouse, tumors and vital organs were harvested and cultured for bacterial numbers at 24, 48, 72 and 96 hours after oral administration. As shown in FIGS. 1A and 1B, PLZ4-Salmonella homing to the tumor was much higher than to any other organs. Most bacteria entering from the indigestive system into the body go to the liver because of the portal vein system. In this experiment, the colonies of PLZ4-Salmonella-haPD1-EGFP at tumors were 6,208, 6,123, 920 and 80 times higher than that in the liver at 24, 48, 72 and 96 hours after administration (normalized by the weight of tumors and organs, and calculated at CFU per gram of tissue). Because Salmonella used in this study depended on three sugars to survive and these three sugars did not exist in these mice, a significant decrease in Salmonella colonies were observed at 96 hours. From 72 hours to 96 hours after oral administration, a decrease of 99.6% of PLZ4-Salmonella-PD1/EGFP was observed in tumors.

### Cancer-Specific Delivery of PLZ4-Salmonella Expressing KillerRed, a Red Protein Photosensitizer

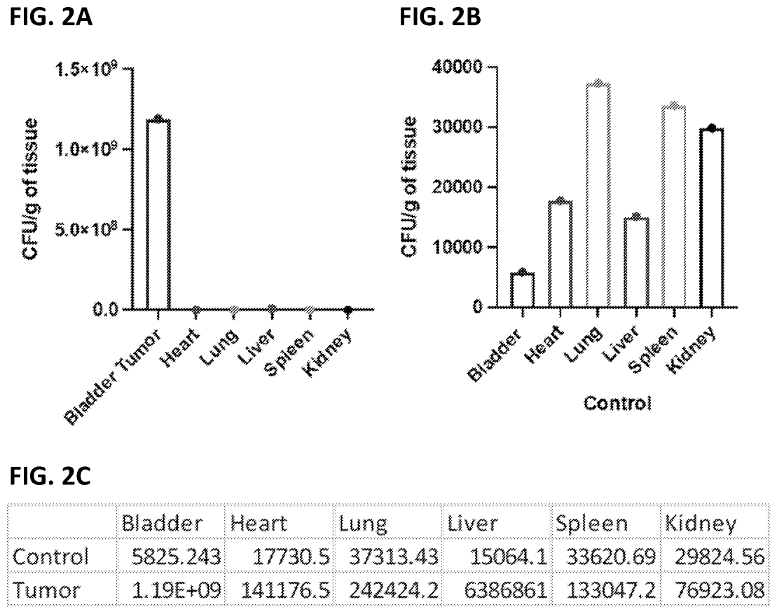

Cancer-specific delivery of PLZ4-Salmonella-KillerRed in SV40T/Ras double transgenic mice was determined. These mice developed orthotopic bladder cancer before Day 25. After oral administration of 5×10⁸ PLZ4-Salmonella-KillerRed per mouse, bladder/bladder tumor and vital organs were harvested and cultured for bacterial numbers (FIG. 2). In the SV40T/Ras double transgenic mouse, PLZ4-Salmonella-KillerRed in the bladder tumor (1.19×10⁹ CFU per gram of tissue) was 186 times higher than the liver (6.39×10⁶ CFU per gram of tissue). In the control mouse without bladder tumor, PLZ4-Salmonella-KillerRed in the normal bladder (5,825 CFU per gram of tissue) was the lowest compared to any other major organs, 2.04×10⁵ times lower than the PLZ4-Salmonella-KillerRed in the bladder tumor of the transgenic mouse, as expected.

In Vivo Distribution of Control Salmonella without PLZ4 on the Surface

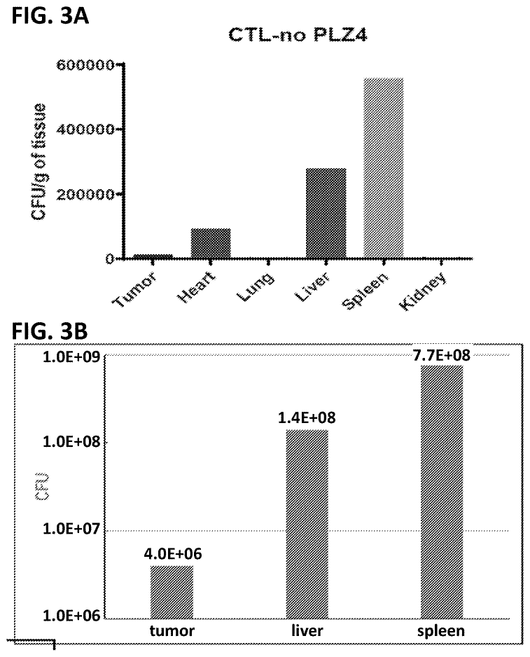

The distribution after oral administration of a control Salmonella without PLZ4 on the surface was also determined. Mice carrying subcutaneous bladder cancer UPPL implants were used for this experiment. Since 72 hours is the peak time for Salmonella distribution, the Salmonella distribution at 72 hours after oral administration was used for comparison. Salmonella in the liver and spleen was 23 and 46 times higher than that in tumors, suggesting that Salmonella homing to tumors was mediated by the PLZ4 cancer-targeting ligand on the surface (FIG. 3A). Because Salmonella from the digestive system go to the liver first before going to other organs, the homing of control Salmonella (without PLZ4 on the surface) after intravenous administration was determined to see if more Salmonella could be found in tumors. Control Salmonella at tumors were 35 and 192.5 times lower than that in the liver and spleen after intravenous administration (FIG. 3B).

### PLZ4-Salmonella can Express Genes of Interest at the Cancer Sites in a Subcutaneous Tumor Model

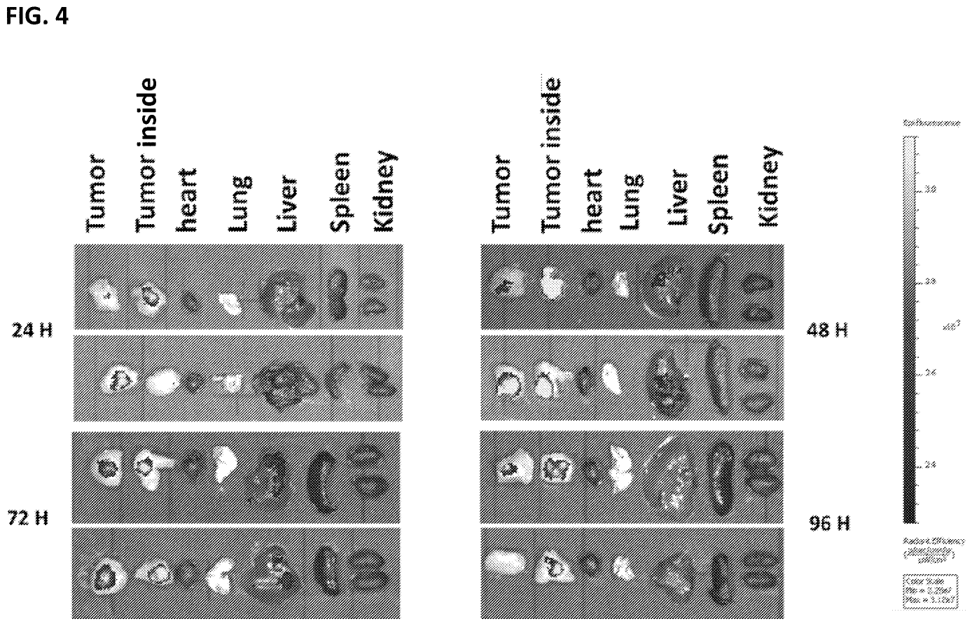

Next, whether PLZ4-Salmonella could express genes of interest inside tumors after it homed to cancers was determined. For this experiment, PLZ4-Salmonella expressing KillerRed, a red fluorescent protein was used. First, mice carrying subcutaneous bladder cancer syngeneic UPPL bladder tumor model were used. UPPL syngeneic bladder cancer cells (5×10⁶ cells) were injected to the flank. When tumors were palpable, mice were fed with used PLZ4-Salmonella-KillerRed (5×10⁸ CFU per mouse) at 24, 48, 72 and 96 hours before euthanasia for imaging. In this experiment, red fluorescence was clearly observed in tumors with very little fluorescence in other organs (FIG. 4). The fluorescence peaked around 48-72 hours. Some red fluorescence was observed in the liver.

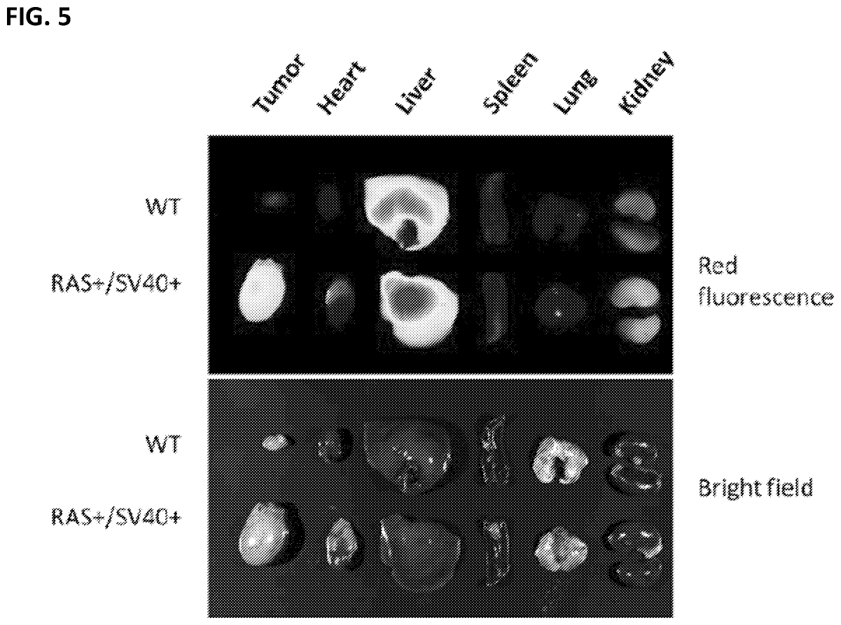

Next, an SV40T/Ras double transgenic mice which develop orthotopic bladder cancer within one month of age was used. When bladder cancer was palpable, a control mouse without bladder cancer and a double transgenic mouse carrying an orthotopic bladder cancer received PLZ4-Salmonella-KellerRed at 5×10⁸ CFU each. Both mice were sacrificed at 72 hours after oral administration. Vital organs, including the bladder, were harvested for detection of red fluorescence. Fluorescence was observed in the liver and orthotopic bladder cancer, but not in other organs, including the normal bladder (FIG. 5). In this figure, the fluorescence in the bladder cancer was weaker than that in the liver. This may be because bladder cancer developed in the urothelial layer within the bladder and was covered by the bladder muscle layer in the bladder wall while there was no other tissue to cover the liver and fluorescence in the liver was directly detected. When both the bladder cancer and the liver were homogenized and cultured for Salmonella, Salmonella in the bladder cancer was 186 times higher than that in the liver (1.189×10⁹ CFU per gram in the bladder cancer versus 6.387×10⁶ CFU in the liver versus 5,825 CFU in the bladder).

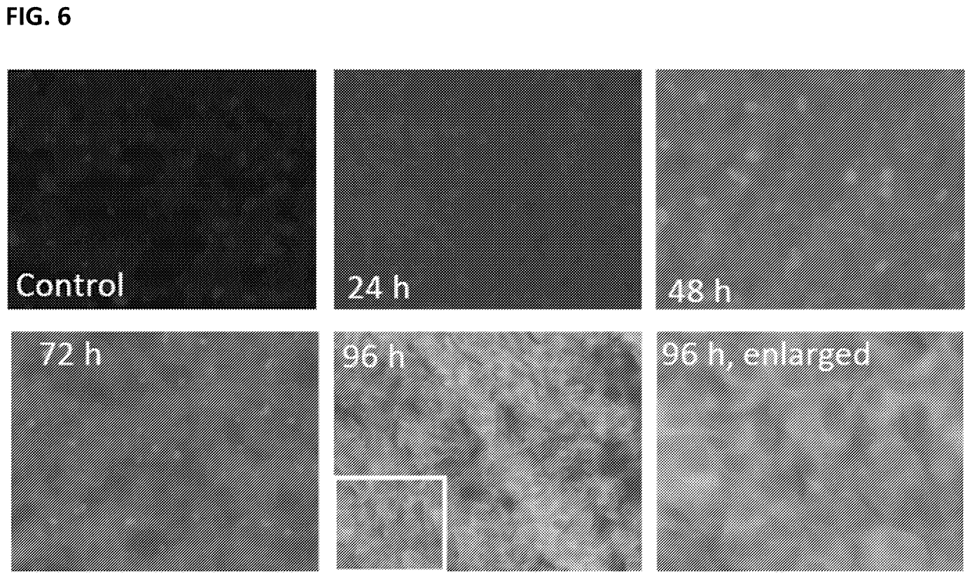

PLZ4-Salmonella was used to deliver another chimeric protein in which a high-affinity Programmed Cell Death 1 (haPD1) conjugated by enhanced green fluorescent protein (EGFP) at the C-terminus (haPD1-EGFP). C57BL/6 mice carrying UPPL subcutaneous tumors were fed with PLZ4-Salmonella-haPD1-EGFP at 24, 48, 72 and 96 hours prior to euthanasia. Tumors were harvested and freshly frozen and sliced for detection of EGFP expression (green fluorescence) (FIG. 6). All the tumor slices had the same 140 milli-second exposure under the same condition. No fluorescence was observed in the control, suggesting there was no background auto-fluorescence. Green fluorescence slightly increased at 24 hours. At 48 and 72 hours after oral administration of PLZ4-Salmonella-haPD1-EGFP, significantly more intense green fluorescence was observed with aggregates and this was consistent with high Salmonella density at these two time points as shown in FIG. 1. The fluorescence intensity was the highest at 96 hours with no visible aggregates. Enlargement of the imaging suggested that green fluorescence is largely on cell membrane in addition to at the extra-cellular areas. This was consistent with the fact that haPD1 binds to Programmed Death Ligand 1 (PD-L1) on cancer cell membrane.

### PLZ4-Salmonella Expressed Genes of Interest Locally at the Cancer Sites to Induce Pharmacological Effects

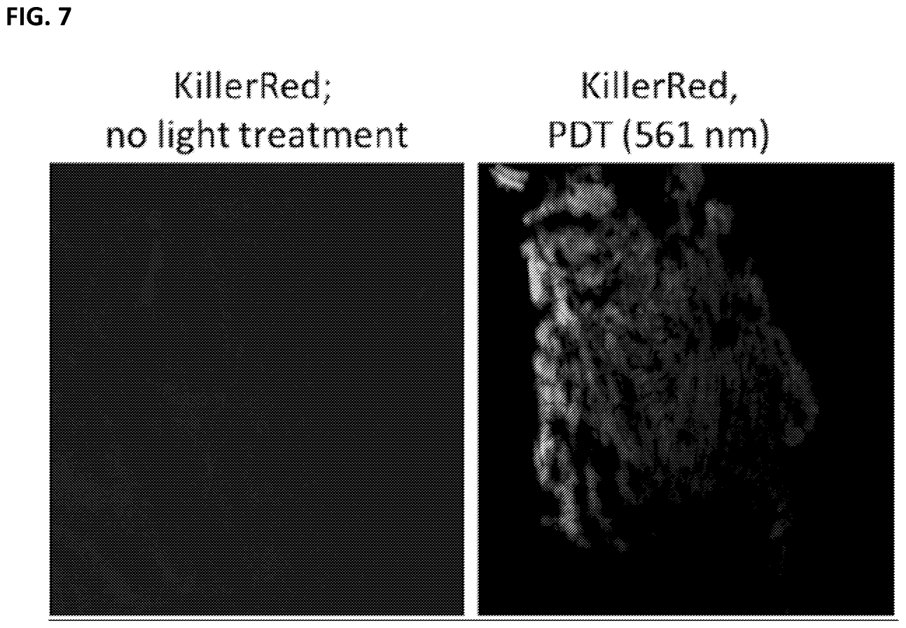

To determine whether oral administration of PLZ4-Salmonella could deliver genes of interest and express proteins of interest locally at the cancer site at a sufficiently high concentration to induce pharmacological response. First, whether KillerRed expressed by PLZ4-Salmonella-KellerRed can be activated by light to produce reactive oxygen species (ROS) and kill cells, a process called photodynamic therapy (PDT) was determined. To confirm whether KillerRed produced by PLZ4-Salmonella-KellerRed can be activated by light for PDT, two C57BL/6 mice carrying UPPL syngeneic bladder cancers were fed with PLZ4-Salmonella-KellerRed at 5×10⁸ CFU per mouse. After 72 hours, laser (wavelength: 561 nm; 100 mW for 20 minutes) was applied to the tumor for PDT. After 24 hours, tumors from both mice were harvested, fresh frozen, cut at 30 μm thick, and stained with dihydroethydine (DHE) at 10 μmol/L dissolved in DMSO, and incubated in light-protected humidified chamber at 37 °C for 15 minutes. ROS production (green fluorescence) could only be detected when mice were fed with PLZ4-Salmonella-KellerRed and treated with light (FIG. 7 right panel), but not in the tumor from the mice fed with PLZ4-Salmonella-KellerRed, but not treated with light (FIG. 7 left panel).

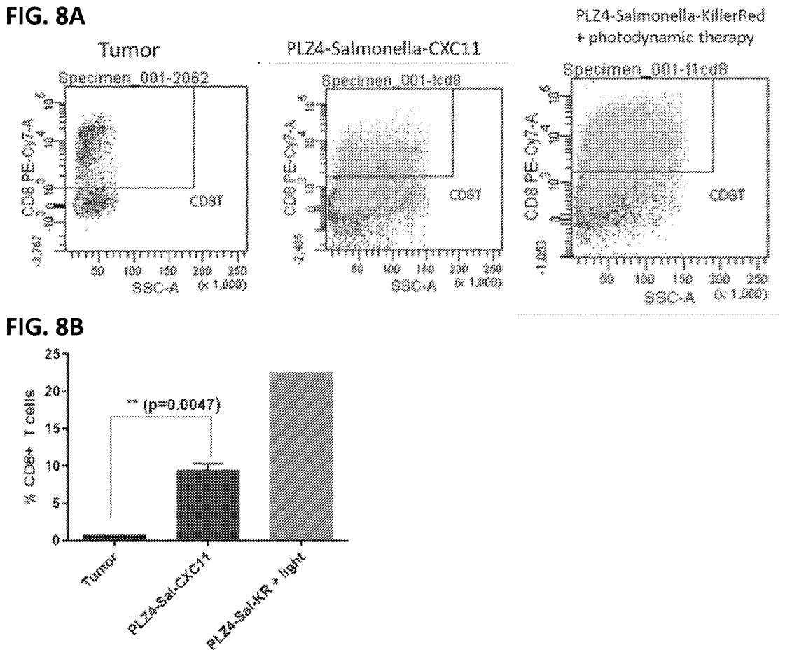

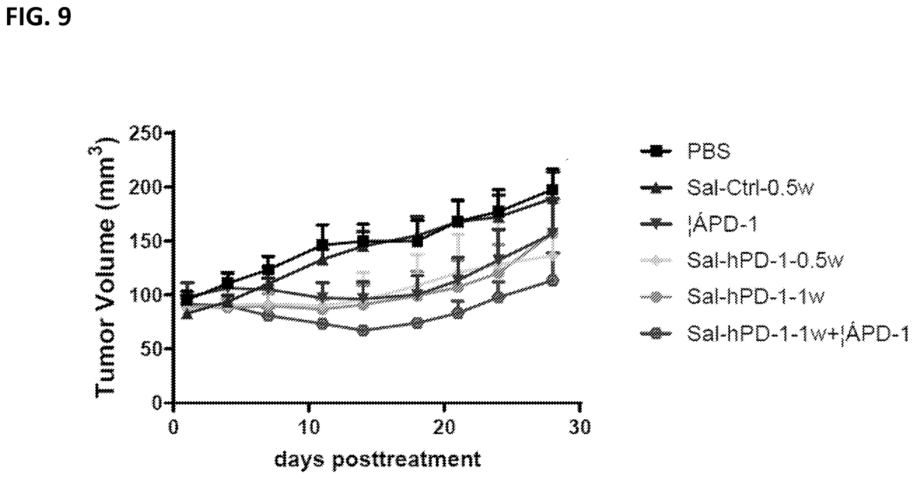

During PDT, ROS can modify proteins and make them immunogenic to stimulate immune response. Dead and dying cells can also stimulate immune response. Consistent with the finding that porphyrin-based PDT can induce immune cell infiltration into tumors and stimulate anti-cancer immunity, FIGS. 8A and 8B show that after PDT, 22.6% of cells in a tumor were T cells, compared to only 0.8% of cells in a control mouse.

Next, PLZ4-Salmonella was used to express CXCL11. CXCL11 is a chemokine that can attract CD8 cytotoxic T cells. For this experiment, C57BL/6 mice carrying syngeneic subcutaneous UPPL bladder cancer implants were used. In the experiment with PLZ4-Salmonella-CXCL11, tumors were harvested 72 hours after oral administration of Salmonella. A significant increase of CD8 T cells inside tumors was observed. Only 0.8% of the cells in the control tumors were CD8 T cells. In contrast, 9.5% of cells in the tumors from mice treated with PLZ4-Salmonella expressing CXCL11 were T cells, an 11.9-fold increase (p=0.0047) (FIGS. 8A and 8B). This suggests that PLZ4 can not only guide the delivery of Salmonella to cancer sites, but more importantly, Salmonella can express sufficiently high concentrations of biologically active proteins at the cancer sites that can induce biological effects.

PLZ4-Salmonella Expressing haPD1-IgG2a Induced Tumor Response after Oral Administration

In this experiment, whether oral PLZ4-Salmonella could deliver high-affinity PD1 that can be used for cancer immunotherapy was determined. There are several major issues associated with current intravenous immunotherapy with immune checkpoint inhibitors anti-PD1/PD-L1 antibodies: after intravenous administration, non-specific delivery of anti-PD1/PD-L1 antibodies to non-cancerous tissues induces immune response to those non-cancerous tissues and causes immune mediated side effects; intravenous administration is inconvenient and expensive; antibody production, purification, transportation and storage are costly; and anti-PD1/PD-L1 antibodies are not very effective with the response rate of around 20-30%. If oral PLZ-Salmonella can deliver immune checkpoint antibodies, cancer-specific delivery can possibly reduce or eliminate immune-mediated toxicity; it is convenient with oral administration; and PLZ4-Salmonella is cheap and fast during production. In this experiment, a high-affinity PD1 fragment (Maute et al. Proc Natl Acad Sci USA. 2015; 112: E6506) was conjugated to the Fc portion of mouse IgG2a. This chimeric gene was then transformed into PLZ4-Salmonella.

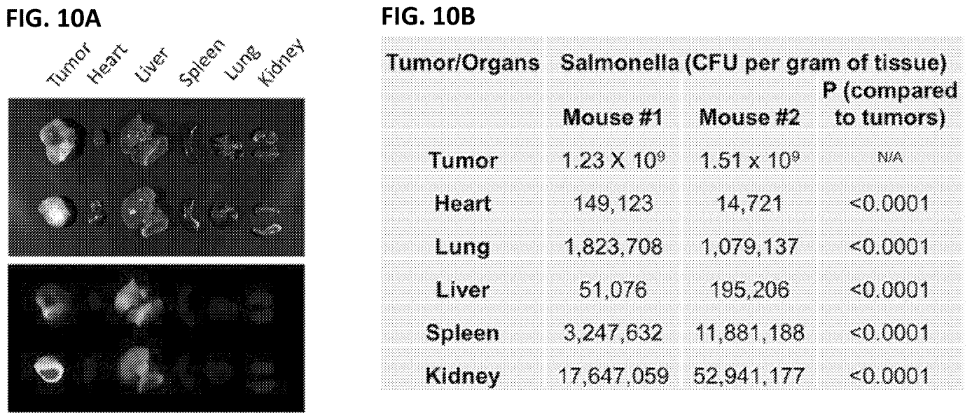

C57BL/6 mice carrying syngeneic BBN963 bladder cancer implants were used for this study. When the tumors reached the size of around 50-100 mm³, mice were randomized to the following treatment: phosphate-buffered solution (PBS) control, control Salmonella without PLZ4 (5×10⁸ CFU, oral gavage once weekly), anti-PD1 antibody (BioXcell, Cat #BE0146, Lebanon, NH. 200 μg intravenous twice weekly); PLZ4-Salmonella-haPD1-IgG2a (5×10⁸ CFU, oral gavage twice weekly); PLZ4-Salmonella-haPD1-IgG2a (5×10⁸ CFU, oral gavage once weekly); and the combination of anti-PD1 antibody and PLZ4-Salmonella-haPD1-IgG2a (5×10⁸ CFU, oral gavage once weekly). As shown in FIG. 10, PLZ4-Salmonella-haPD1-IgG2a, once or twice weekly, was as effective as the anti-PD1 antibody in inducing anti-tumor activity. However, the combination therapy was even more effective.

PLZ4-Salmonella could Still Target and Home to Cancer Sites in Mice Previous Immunized with Salmonella

Whether prior immunization of Salmonella inhibited subsequent cancer homing of PLZ4-Salmonella was determined. Cancer patients usually need long-term frequent treatment. Salmonella can induce immune response that can subsequently suppress cancer targeting of future administration of Salmonella. To determine whether primary immunization of Salmonella inhibited future cancer homing, C57BL/6 mice were immunized with Salmonella at 5×10⁸ CFU per mouse on Day 1, 4, 22, 24, 36 and 38. On Day 38, syngeneic bladder cancer cells BBN963 cells was inoculated at the flank. When tumors reached around the size of 1.5 cm³ (around 90 days), mice received PLZ4-Salmonella expressing KillerRed 72 hours prior to euthanasia. Tumors and vital organs were harvested for imaging and determination of Salmonella homing (FIGS. 10A and 10B). Salmonella in the liver in both mice was much higher than in any other organs (p<0.0001).

### LHRH can Guide Cancer-Specific Delivery to Breast Cancer Cells

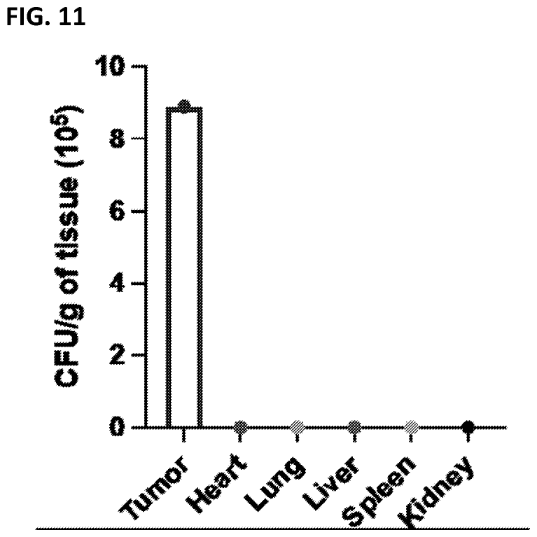

Another targeting peptide, Luteinizing hormone-releasing hormone (LHRH), was tested to determine whether it can guide cancer-specific delivery to cancer cells. LHRH receptor was found to be overexpressed on several cancer types, such as breast cancer, prostate cancer and bladder cancer. In this experiment, the LRHR peptide (amino acid sequence: QHWSYGLRP) is expressed on the third loop of Salmonella OmpA. In this experiment, breast cancer cell line MDA-MB-231 cells were injected into immunocompromised NSG mice. When the tumor reached around 120 mm³, 0.36×10⁸ CFU of LHRH-Salmonella was administrated through oral gavage. At 72 hours, tumor and vital organs were harvested, homogenized and cultured for bacterial colonies. LHRH-Salmonella was observed in the tumor only (FIG. 11).

### Example 2: PLZ4-Salmonella Delivers High-Affinity PD1 and Exerts Anti-Cancer Effects

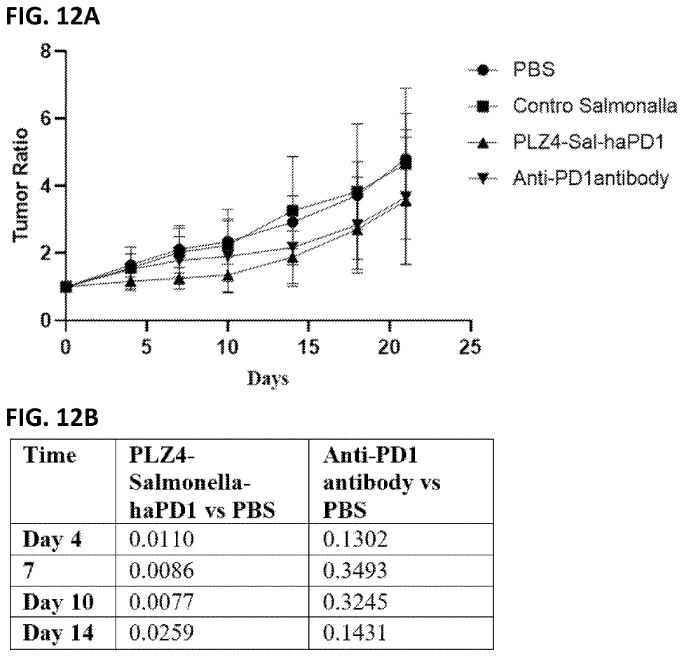

C57BL/6 mice carrying syngeneic BBN963 bladder cancer implants were used for this study. When the tumors reached a size of around 50-100 mm³, mice were randomized to the following treatment: phosphate-buffered solution (PBS) control, control Salmonella without PLZ4 (10⁹ CFU, oral gavage once weekly), anti-PD1 antibody (BioXcell, Cat #BE0146, Lebanon, NH. 200 μg intravenous once weekly), or PLZ4-Salmonella-haPD1-IgG2a (10⁹ CFU, oral gavage once weekly). As shown in FIG. 12A, PLZ4-Salmonella-haPD1-IgG2a significantly reduced tumor growth starting at the first tumor measurement of Day 4. Anti-PD1 antibody reduced tumor growth, but the tumor reduction did not reach statistical significance (FIG. 12B). Control Salmonella had no effect on tumor growth.

### Example 3: PLZ4 Guides Cancer-Specific Delivery of Listeria

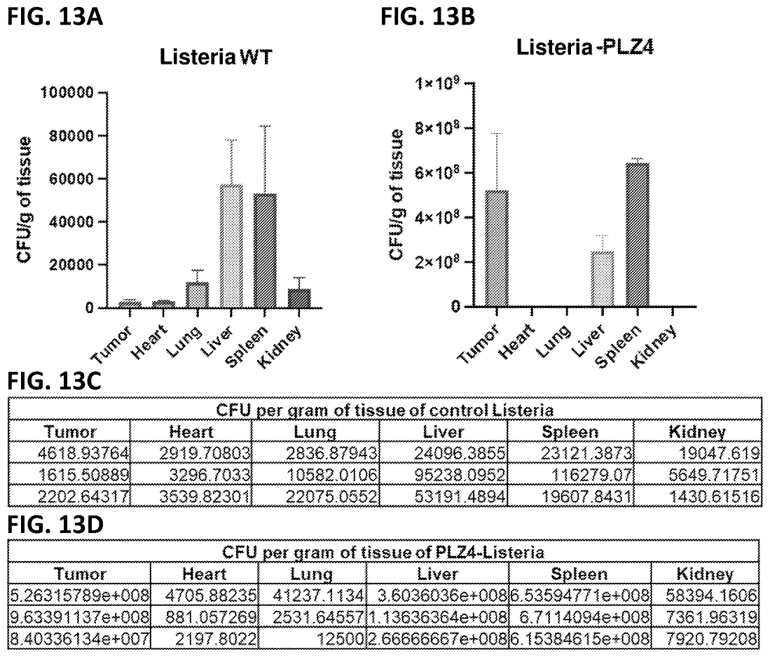

To determine whether PLZ4 could guide cancer-specific delivery of other bacteria, PLZ4 was expressed on the surface of attenuated Listeria monocytogenes. Specifically, PLZ4 was expressed on the third loop of OmpA on the surface of Listeria monocytogenes. C57BL/6 mice carrying syngeneic BBN963 bladder cancer implants were used for this study. When the tumors reached a size of over 100 mm³, mice were fed with oral gavage of control Listeria or PLZ4-Listeria at 10⁸ CFU per mouse. Mice were euthanized and organs were harvested around 72 hours after feeding. The tumors and organs were homogenized and cultured for Listeria. As shown in FIGS. 13A-13D, the control Listeria in the liver was 5-59 times of the bacteria in the bladder tumors while PLZ4-Listeria in tumors was 1.46-8.45 times of the bacteria in the liver, showing that PLZ4 can guide cancer-specific delivery of bacteria aside from Salmonella.

EXEMPLARY SEQUENCES
SEQ ID NO: 1
X₁DGRX₅GF,
wherein X1 and X5 are A, D, E, F, G, H, I, K,
L, M, N, P, Q, R, S, T, V, W, or Y
SEQ ID NO: 2
QDGRMGF
SEQ ID NO: 3
QDGRKGF
SEQ ID NO:4
QDGRKGGF
SEQ ID NO: 5
CX₁DGRX₅GFC,
wherein X1 and X5 are A, D, E, F, G, H, I, K,
L, M, N, P, Q, R, S, T, V, W, or Y
SEQ ID NO: 6
CQDGRMGFC
SEQ ID NO: 7
QHWSYGLRP
SEQ ID NO: 8
mkdltvlsrwryswrilddneaqkmkktaiaiavalagfatvaqaapkdn
twyagaklgwsqyhdtgfinndgpthenqlgagafggyqvnpyvgfemgy
dwlgrmpykgdningaykaqgvqltaklgypitddldvytrlggmvwrad
tksnvpgQDGRMGFgpstkdhdtgvspvfaggieyaitpeiatrleyqwt
nnigdantigtrpdngllsvgvsyrfgqqeaapvvapapapapevqtkhf
tlksdvlfnfnkstlkpegqqaldqlysqlsnldpkdgsvvvlgftdrig
sdaynqglsekraqsvvdyliskgipsdkisargmgesnpvtgntcdnvk
praalidclapdrrveievkgvkdvvtqpqa
SEQ ID NO: 9
mkdltvlsrwryswrilddneaqkmkktaiaiavalagfatvaqaapkdn
twyagaklgwsqyhdtgfinndgpthenqlgagafggyqvnpyvgfemgy
dwlgrmpykgdningaykaqgvqltaklgypitddldvytrlggmvwrad
tksnvpgQDGRKGFgpstkdhdtgvspvfaggieyaitpeiatrleyqwt
nnigdantigtrpdngllsvgvsyrfgqqeaapvvapapapapevqtkhf
tlksdvlfnfnkstlkpegqqaldqlysqlsnldpkdgsvvvlgftdrig
sdaynqglsekraqsvvdyliskgipsdkisargmgesnpvtgntcdnvk
praalidclapdrrveievkgvkdvvtqpqa
SEQ ID NO: 10
mkdltvlsrwryswrilddneaqkmkktaiaiavalagfatvaqaapkdn
twyagaklgwsqyhdtgfinndgpthenqlgagafggyqvnpyvgfemgy
dwlgrmpykgdningaykaqgvqltaklgypitddldvytrlggmvwrad
tksnvpgQDGRKGGFgpstkdhdtgvspvfaggieyaitpeiatrleyqw
tnnigdantigtrpdngllsvgvsyrfgqqeaapvvapapapapevqtkh
ftlksdvlfnfnkstlkpegqqaldqlysqlsnldpkdgsvvvlgftdri
gsdaynqglsekraqsvvdyliskgipsdkisargmgesnpvtgntcdnv
kpraalidclapdrrveievkgvkdvvtqpqa
SEQ ID NO: 11
mkdltvlsrwryswrilddneaqkmkktaiaiavalagfatvaqaapkdn
twyagaklgwsqyhdtgfinndgpthenqlgagafggyqvnpyvgfemgy
dwlgrmpykgdningaykaqgvqltaklgypitddldvytrlggmvwrad
tksnvpgCQDGRMGFCgpstkdhdtgvspvfaggieyaitpeiatrleyq
wtnnigdantigtrpdngllsvgvsyrfgqqeaapvvapapapapevqtk
hftlksdvlfnfnkstlkpegqqaldqlysqlsnldpkdgsvvvlgftdr
igsdaynqglsekraqsvvdyliskgipsdkisargmgesnpvtgntcdn
vkpraalidclapdrrveievkgvkdvvtqpqa
SEQ ID NO: 12
mkdltvlsrwryswrilddneaqkmkktaiaiavalagfatvaqaapkdn
twyagaklgwsqyhdtgfinndgpthenqlgagafggyqvnpyvgfemgy
dwlgrmpykgdningaykaqgvqltaklgypitddldvytrlggmvwrad
tksnvpgQHWSYGLRPgpstkdhdtgvspvfaggieyaitpeiatrleyq
wtnnigdantigtrpdngllsvgvsyrfgqqeaapvvapapapapevqtk
hftlksdvlfnfnkstlkpegqqaldqlysqlsnldpkdgsvvvlgftdr
igsdaynqglsekraqsvvdyliskgipsdkisargmgesnpvtgntcdn
vkpraalidclapdrrveievkgvkdvvtqpqa

1. A method of delivering a tumor-targeting bacterium to a tumor site in an individual having cancer, comprising orally administering to the individual an effective amount of the tumor-targeting bacterium, wherein the tumor-targeting bacterium comprises on its surface a tumor-targeting agent, wherein upon administration the tumor-targeting bacterium is specifically targeted to the tumor site.

2. The method of claim 1, wherein the tumor-targeting bacterium further comprises a therapeutic or diagnostic agent or a nucleic acid encoding the therapeutic or diagnostic agent, wherein upon administration the tumor-targeting bacterium is specifically targeted to the tumor site, thereby delivering the therapeutic or diagnostic agent to the tumor site.

3. The method of claim 1, wherein the tumor-targeting agent is a tumor-targeting peptide or tumor-targeting protein.

4. The method of claim 3, wherein the tumor-targeting bacterium is genetically engineered to express the tumor-targeting peptide or tumor-targeting protein on its surface.

5. The method of claim 1, wherein the tumor-targeting agent is attached to the surface of the tumor-targeting bacterium.

6. The method of claim 3, wherein the tumor-targeting peptide is no longer than 25 amino acids in length.

7. The method of claim 6, wherein the tumor-targeting peptide is selected from the group consisting of a PLZ peptide and a luteinizing hormone releasing hormone (LHRH) peptide.

8. The method of claim 3, wherein the tumor-targeting peptide comprises the amino acid sequence X₁DGRX₅GF (SEQ ID NO: 1), wherein X₁ is selected from Gln and Gly and X₅ is any amino acid other than cysteine.

9. The method of claim 3, wherein the tumor-targeting peptide comprises the amino acid sequence CX₁DGRX₅GFC (SEQ ID NO: 5), wherein X₁ is selected from Gln and Gly and X₅ is any amino acid other than cysteine.

10. The method of claim 3, wherein the tumor-targeting peptide is embedded in a bacterial protein presented at the outer wall of the tumor-targeting bacterium.

11. The method of claim 1, wherein the tumor-targeting bacterium is Salmonella or Listeria.

12. The method of claim 2, wherein the therapeutic or diagnostic agent is selected from the group consisting of: a protein (such as chemokine e.g., CXCL11), an antibody, an mRNA, an siRNA, an antisense RNA, and a CRISPR/gRNA system.

13. The method of claim 12, wherein the therapeutic agent is an immune check point inhibitor, optionally selected from the group consisting of PD1-Fc, PD-L1-Fc, anti-PD1 antibody, and an anti-PD-L1 antibody.

14. The method of claim 12, wherein the therapeutic agent is a toxin or a photosensitizer.

15. The method of claim 1, wherein the tumor-targeting bacterium is administered at about 1×10⁷ to 5×10¹² Colony forming units (CFU) per dose.

16-17. (canceled)

18. The method of claim 1, wherein the individual has breast cancer, prostate cancer, or bladder cancer.

19. The method of claim 1, wherein the individual has a healthy gut microbiome.

20. The method of claim 1, wherein the individual is a human individual.

21. An oral formulation of tumor-targeting bacterium comprising on its surface a tumor-targeting agent and a pharmaceutically acceptable excipient.

22-38. (canceled)
